(19) United States
(12) Patent Application Publication
(10) Pub. No.: US 2012/0231528 A1
(43) Pub. Date: Sep. 13, 2012

(54) REACTION CASING FOR A PHOTOSYNTHETIC REACTOR AND ASSOCIATED PHOTOSYNTHETIC REACTOR

(75) Inventors: Arnaud Muller-Feuga, Baillargues (FR); Michel Lemar, Saint Symphorien d'Ozon (FR)

(73) Assignee: MICROPHYT, Baillargues (FR)

(21) Appl. No.: 13/508,281

(22) PCT Filed: Nov. 8, 2010
(86) PCT No.: PCT/FR2010/052392

§ 371 Date: May 4, 2012

Related U.S. Application Data
(60) Provisional application No. 61/259,704, filed on Nov. 10, 2009.

(30) Foreign Application Priority Data
Nov. 10, 2009 (FR) 0957933

(51) Int. Cl. C12N 1/12; B01L 1/00; B23P 11/00; C12M 1/42

(52) U.S. Cl. 435/257.1; 435/292.1; 422/565; 29/428

(57) ABSTRACT

“A reaction casing for a photosynthetic reactor designed firstly to float on a body of water and secondly to delimit a biphasic flow pathway for gas/liquid culture medium between a first and a second opening of the casing, where the casing includes two claddings, respectively outer and inner, made at least in part of a material transparent to light rays, the inner cladding extending inside the outer cladding so that the claddings delimit between them an inter-cladding space in fluid connection with the first opening of the casing, the outer cladding has an open proximal end and a closed distal end and in the inner cladding has an open proximal end in fluid connection with the second opening of the casing and a distal end provided with at least one communication orifice between the inside of the inner cladding and the inter-cladding space.”

### TECHNICAL FIELD

The present invention relates to a reaction casing for a photosynthetic reactor adapted for the culture of photosynthetic microorganisms, algae in particular, to a method for fabricating said reaction casing, to an associated photosynthetic reactor and also to a culture method of photosynthetic microorganisms using said reactor.

It relates more particularly to a reaction casing designed firstly to float on a body of water and secondly to delimit a gas/liquid culture medium biphasic flow pathway between a first and a second opening of the said reaction casing.

The present invention relates to the culture of any photosynthetic organism i.e. any form of life able to develop and photosynthesize in a suitable nutrient culture medium, in the presence of sunlight and carbon-rich gas such as carbon dioxide, microalgae being the main representatives of this form of life.

### BRIEF DISCUSSION OF RELATED ART

The analysis of compared performance levels of photosynthetic organisms leads to giving priority to the culture of microalgae which are the oldest players in photosynthesis on our planet. Unlike upright plants they do not have any complex member that is long to construct in order to access water and light. They do not need to rigidify their stem by means of metabolites (cellulose, lignin) which degrade with difficulty. The result is increased efficacy of microalgae cultures translating as surface productivities possibly reaching 100 tons (dry weight) per hectare and per year, compared with 10 tons for the best plants in open fields. All the biomass can be put to use whereas the harvesting of major cultures generally only concerns the grains with the notable exception of sugar cane and animal feed plants which at least leave behind the root system.

Among the photosynthetic microorganisms concerned by the invention are more particularly included aquatic plants such as microalgae, moss protonemata, small macro-algae and the isolated cells of multicellular plants. These aquatic plants have properties of interest particularly in the fields of pharmacy, human and animal nutrition, dermo-cosmetology, energy and the environment.

As for most photosynthetic microorganisms, the access to this resource essentially comprises assisted culture in adapted reactors. Since light is their main substrate, the culture medium must have an optical interface receiving a flow of light. The difficulty of photosynthetic microorganism culture lies in the fact that they themselves form obstacles to the passing of light which is their main substrate. The growth of the culture will therefore stabilize when the light can no longer enter into the thickness of the culture. This phenomenon is known as self-shading.

The [symbol] light path length [symbol] allows the characterizing of the different confinement modes, and is defined as:

the length of the light path from its entry into the culture via a transparent optical interface as far as an opposite opaque wall; or

one half of the distance which separates the two transparent optical interfaces when the confinement receives light via two opposite, transparent optical interfaces.

This light path length varies between a few centimeters to a few decimeters and mostly determines the production of biomass per unit of time and per unit of optical surface (surface productivity in g/m²/j) and the concentration of the culture (en g/L) in the final growth phase. The different confinement modes used to ensure the culture of small aquatic plants can therefore be classified in relation to this characteristic length.

The photosynthesis reaction is also accompanied by consumption of carbon dioxide (CO₂) and production of oxygen (O₂). Excess oxygen inhibits the reaction, whilst the absence of carbon dioxide interrupts the reaction for lack of substrate to be converted. A gas/liquid interface must therefore be provided for the transfers of mass between these gases and the liquid phase. To promote these exchanges and to avoid heterogeneities, the culture must be the seat of a mixture intended to renew the organisms at the above-mentioned optical interface and also at this gas/liquid interface.

One first known embodiment of a photosynthesis reactor consists of an open container of pond or vessel type in which the culture is held under gravity and has a free surface which alone forms the optical interface and the gas/liquid interface. The culture is mixed inside the pond by one or more mechanical mixer devices e.g. of paddle wheel type. The pond cultures thus formed may cover major surface areas and this embodiment lies at the base of most current world production of microalgae, which reaches several thousand tons in dry weight. The photosynthetic organisms produced by this type of reactor are essentially:

so-called extremophilic algae whose media are hostile to predators and competitors, such as the algae of spirulin or Dunaliella type; or

so-called dominant algae which withstand mechanical stresses or contaminations better than the others, such as for example the algae of type Chlorella, Scenedesmus, Skeletonema, Odontella or Nannochloropsis.

A second known embodiment of photosynthetic reactor also consists of an open container of reservoir or vessel type but whose dimensions are smaller than those of the ponds in the first known embodiment. These containers generally have side walls transparent to sunlight so that the optical interface is formed both by the free surface of the liquid medium and by the transparent side walls.

In this second embodiment, it is conventional to have recourse to an injection of air made into the lower part of the reservoir which leads to the formation of air bubbles rising in the liquid as far as the free surface. The surface of the air bubbles thus formed forms the gas/liquid interface. When rising to the surface, the bubbles carry the culture upwards thereby creating convective movements which may extend to the entire volume. Carbon dioxide (CO₂) is sometimes added to the injected air to provide a carbon excess as per a predefined molar fraction of a few percent.

Of smaller volume than the ponds of the first embodiment, the reservoirs of the second known embodiment are adapted to more controlled cultures, in particular to cultures of microalgae intended for the feeding of shellfish larvae or as living prey for fish larvae in aquaculture. The frequent cleaning of these reservoirs and pure, massive inoculations allow contaminations inside to the reservoir to be limited. Numbering several tens of species the microalgae thus cultivated have relatively close needs in terms of temperature and light making it possible for them to be cultured in common locations.

These two embodiments in the form of an open vessel offer a light path length of one to several decimeters.

A third known embodiment of photosynthetic reactor consists of a closed reactor called a photobioreactor which comprises a closed loop inside which the liquid culture medium circulates, the said closed loop comprising a reaction pipe provided with reaction sections made in material transparent to light rays (or to light), and a return pipe ensuring the connection between the two opposite ends of the reaction pipe.

Photobioreactors, described in particular in documents GB 2 118 572 A, ES 2 193 860 A1, GB 2 331 762 A, ES 2 150 389 A1, FR 2 685 344 A1 et FR 2 875 511 A3, offer substantially shorter light path lengths of the order of one to several centimeters compared with the open pond embodiments, and they allow concentrations of photosynthetic organisms to be reached of several grams per liter protected from air contamination. The reaction pipe of photobioreactors generally consists of transparent flat plates or of tubes in glass or plastic material with a thickness or diameter of the order of one centimeter and connected end to end by elbows to form together a channel of coiled shape.

The return pipe comprises a vertical so-called riser tube in which the liquid medium moves upwards and a downcomer tube in which the liquid medium descends under the effect of gravity.

The gas injection system generally used in photobioreactors consists of a gas-lift [symbol] i.e. the injection of gas at the base of the vertical riser tube of the return pipe; the said gas injection being used both to circulate or move the liquid reaction medium and to achieve gas-liquid exchanges. The gas-lift in its upper part comprises a header tank of larger volume in which slower rates of circulation allow gas-liquid separation and the downcomer vertical tube of the return pipe leads into the bottom of the header tank to feed liquid to the reaction pipe.

The above-mentioned photobioreactors apply the principle according to which the reaction only takes place in the liquid phase, in other words these photobioreactors seek to minimize the volume of gas injected into the reactor so as not to reduce the volume of liquid culture medium accordingly for the purpose of avoiding a decrease in production. Therefore in these photobioreactors, the extraction of oxygen is obtained by the vertical riser tube defined above; the said vertical riser tube forming a column of air bubbles opening into the header tank receiving the liquid culture medium, and comprising an injection of gas at its lower part, expediently air enriched with CO₂. As described above, the two functions of gas circulation and transfer are merged within this single device called a gas-lift which creates upward vertical circulation by exchange of quantity of movement between the liquid mass and the gas bubbles resulting from injection. The photosynthetic oxygen supersaturated in the liquid passes into the gas phase by air scavenging, whilst the CO₂ passes into solution. These functions of degassing and carbonization are indispensable and occur simultaneously and inseparably in this single device in which the culture must have high frequency passing to avoid a deleterious increase in the content of dissolved oxygen.

Gas lifts have the disadvantage of generating gas bubbles which rise in the vertical riser tube of the return pipe of photobioreactors. The Applicant has effectively observed the deleterious role of these bubbles for the culture of microorganisms in photobioreactors:

firstly, the bubbles place mechanical stress on the microalgae and may harm fragile microorganisms; and

secondly, by surfactant effect the bubbles capture the molecules which have surface active properties, and in particular organic molecules, cell debris and excretion products of living cells. These substances, normally dispersed in the medium in the absence of bubbles, are therefore grouped together in the form of aggregates on the free surface of the header tank when the bubbles burst. The bacteria and fungi which could not develop on account of the strong dilution of these organic molecules then find concentrated substrates favorable for their development.

The invention avoids or at least limit the formation of bubbles in order to:

contain bacterial and fungal development for example to remain compatible with health standards conventionally laid down for microorganism culture; and

limit mechanical stresses in the liquid culture medium and thereby allow the culture of some fragile microorganisms which up until now have been excluded from said reactor culture.

In one embodiment alternative to a gas lift, the deoxygenation of the liquid culture medium circulating in the photobioreactor is obtained by causing the liquid medium to drop under gravity into a container of constant level. The liquid culture medium is set in circulation here by pumping means, notably of centrifugal pump type, arranged in the reaction tubing designed not only to offset head losses in the tubing but also to raise the culture level accordingly.

Although it generates fewer bubbles, this device with centrifugal pump is as mechanically harmful for microorganisms as the gas-lift. To overcome head losses, on each passing above the pumping means mechanical stresses are generated which may disturb the growth of the microorganisms and cause mortalities within the culture. Production levels are thereby deteriorated, at times prohibitively.

It has been ascertained for example that it is not possible to culture some so-called fragile microalgae in photobioreactors comprising centrifugal pumps to cause the culture to circulate. These fragile microalgae appear to be all the more sensitive to mechanical stresses that they form chains and/or exhibit appendages such as hairs, flagella, and spicules. Some microalgae such as algae of type Haematococcus pluvialis for example lose their flagella and encyst forming a thick resistant cellular wall. On the other hand, other microalgae such as algae of type Chlorella vulgaris or Nannochloropsis oculata for example, do not have an appendage but have a thick cellular wall so that the resist when passing through the pumping means and in particular pumps of centrifugal type.

However, it is difficult to identify the type of mechanical stresses which influence the survival and growth microorganisms. Most authors are in agreement that shearing and accelerations have the most deleterious influence. Shearing sets up tensions which may deteriorate cell integrity with tearing of the wall of the microorganisms and spilling of cytosol. Accelerations deteriorate cell structure through an increase in the gravitational field.

Living cells are ill-prepared for such forces and perhaps even more so aquatic cells which live in hydrostatic equilibrium and have not developed a structure capable of overcoming a gravitation field. In addition, aquatic cells are sensitive to threshold values and probably also to exposure variations and exposure time. In the current state of knowledge, it is difficult to predict the mechanical effects of hydrodynamic conditions imposed upon cells.

### BRIEF SUMMARY

The invention reduces the mechanical effects imposed on microorganisms, in particular effects of shear and acceleration type, so as to extend the number of species which can be cultured inside the reactor to those which are the most sensitive to these harmful mechanical effects, in other words to provide a reactor allowing the culture of fragile microorganisms such as the fragile microalgae mentioned above for example.

In addition, the Applicant has observed that the culture yield of photobioreactors equipped with a gas-lift or centrifugal pump is limited particularly on account of the formation of bubbles. The Applicant has determined that the culture yield partly depends on the phenomena involved in the gas-liquid transfer to avoid losses and to reduce this major expenditure item. Modeling of the gas-liquid transfer of carbon dioxide intended for the reaction and of the oxygen that it produces requires the determination of the rate of transfer which is a function of the surface transfer coefficient.

The surface transfer coefficient is a key parameter translating the performance level of a gas/liquid exchange system in the stable state. This surface transfer coefficient is equal to the product of the volume transfer coefficient of matter towards the liquid [symbol] KL [symbol] (m.s⁻¹) and of the interface surface area in relation to volume [symbol] a [symbol] (m⁻¹), where:

a=(α_{G}.S)/V
a: Interface surface area relative to volume (m⁻¹);
a_{G}: Coefficient of phase retention;
S: Contact surface (m²); and
V: Volume of the reactor (m³).

The surface transfer coefficient therefore depends on the geometry of the gas/liquid exchange system but also on the physicochemical properties of the liquid and gas. For gas/liquid exchange in a vertical column of bubbles, the exchange surface depends on the number of bubbles and their size. The population of bubbles generated by an injection of gas into a liquid depends on the injection flow rate, the geometry of the injector, and the difference in pressure either side thereof.

The invention provides a photosynthetic reactor which allows the mass culture of photosynthetic microorganisms, and the extension thereof to the most fragile species by means of a reactor which meets the following issues:

to reduce, even to avoid, mechanical stresses generally related to agitation and the setting in circulation of the culture medium, which reduce performance levels of survival and growth of photosynthetic microorganisms such as microalgae, more particularly chain-forming microalgae having appendages;

to reduce and even to avoid the production of bubbles of small size likely to promote the aggregating of organic molecules and the development of heterotrophic microorganisms for which they act as substrate;

whilst obtaining photon transfer to deliver sunlight to photosynthetic microorganisms, the mass transfer of gas/liquid transfer being essential for providing carbon and evacuating oxygen, and heat transfer for evacuating the calories brought by light rays, and to hold the culture at the right temperature; and;

whilst maintaining mechanical conditions, to preserve cell integrity by avoiding exchanges with the surrounding natural environment which may lead to contaminations and disseminations.

With regard to the three embodiments described above, their greatest limitation to the development of algae production is due to the fact that they are intended for emerged surfaces of the planet which are used in priority for urbanization and agricultural crops for food, and are becoming increasingly rarer as human demography increases. This lack of surface area severely limits the development of microalgae cultures using such surfaces in particular microalgae that are energy-oriented which need to occupy considerable surface areas in order to play a significant role.

To meet this issue, the production of algae cultures has been envisaged in desert regions, but this perspective is countered by the short availability of water to produce the reaction liquid medium and for cooling thereof by evaporation.

Yet bodies of water such as natural and artificial lakes across continents especially the seas cover the largest surfaces of the Earth and as yet are only put to little use for their light exposure. These tracts of water or aquatic surfaces are a natural site for considerable plant productions which form the first level of aquatic trophic chains partly harnessed by world fisheries. The primary production of the oceans currently estimated at about 10¹¹ tons per year is the most important on the planet. These plant masses are consumed by herbivores as soon as they are produced, which means that they are little visible, sporadic, diluted and difficult to separate from the surrounding mass of water, and are never pure. This explains why this abundant resource is not directly put to use by man.

One of the purposes of the invention is to propose a reaction casing for photosynthetic reactor adapted for the culture of photosynthetic microorganisms, which can be deployed on a body of water or on the surface of lakes and seas. For this purpose the invention proposes using these bodies of water receiving sunlight to ensure, other than the water resource, two essential functions of photobioreactors, namely horizontal buoyancy and heat stability. Several types of photosynthetic reactors deployed on a body of water are known from the state to meet this issue of using expanses of water.

For example, it is known from patent application FR 2 621 323, to provide for a photobioreactor comprising a reaction enclosure in the form of a first assembly of parallel tubes, in flexible plastic material such as polyethylene, connected together at their two ends by means of two collectors respectively upstream and downstream. This first assembly of tubes ensures the confinement of the liquid culture medium. The photobioreactor also comprises a second assembly of tubes placed underneath the first assembly of tubes, in which the tubes of this second assembly are intended to be inflated with compressed air to form a floating pneumatic support. Said photobioreactor has numerous disadvantages of which the chief disadvantages are: a complex, costly reaction enclosure with a succession of tubes and collectors upstream and downstream which make the reaction enclosure cumbersome, and a complex structure intended to ensure the buoyancy of the reaction enclosure on the body of water, required in particular by the presence of these collectors.

Documents FR 2 361 060 and FR 2 324 224 respectively describe a photosynthetic reactor comprising a reaction enclosure in the form of a series of transparent tubes connected together to delimit a continuous flow pathway, of coiled shape, for the liquid culture medium. These tubes are housed in a frame to form a floating structure comprising float housings. Said reactor has numerous disadvantages the main ones being: a complex, costly reaction enclosure with a succession for tubes connected together at their ends, these tubes requiring a complex structure intended to ensure the buoyancy of the assembly.

Document WO 2009/051479 A2 describes a photobioreactor comprising a reactor enclosure in the form of a series of transparent tubes connected together by coupling parts to delimit a continuous flow pathway, of coiled shape, for the liquid culture medium. To ensure the floating of these tubes the photobioreactor comprises floats attached to the tubes. Said photobioreactor has numerous drawbacks, the main drawbacks being: a complex, costly reaction enclosure with a succession of tubes connected together at their ends via coupling parts, these tubes requiring the addition of floats to ensure the buoyancy of the assembly.

Document WO 2008/134010 A2 describes a photobioreactor provided with a reaction enclosure in the form of a tube in flexible transparent material confining the liquid and gas volumes, and with floats arranged on the side of the tube to ensure the buoyancy of the assembly. The deployment of the confinement volume is obtained by means of stiffeners and spacers and the biphasic gas/liquid circulation takes place in only one direction. In this photobioreactor the tube must be connected via its two ends to other installations ensuring the setting in movement and closing of the loop of the reaction liquid.

Document WO 2009/090549 A2 describes a photobioreactor provided with a reaction enclosure in the form of a tubular compartment in flexible transparent material. In this photobioreactor the supply of gas (CO₂) to the liquid culture medium can be made via passive diffusion of gas over a large surface area of the liquid medium by injecting gas bubbles in particular into the lower of the reaction compartment, with all the drawbacks mentioned above relating to the production of bubbles.

The reactors described in the aforementioned documents FR 2 621 323, FR 2 361 060, FR 2 324 224, WO 2009/051479 A2, WO 2008/134010 A2 and WO 2009/090549 A2 have an additional common drawback: the cleaning of the reaction enclosure, inside and outside is particularly complex and requires at least partial dismounting of the enclosure, bearing in mind that the development of dirt and biofilms on the inner or outer walls of the reaction enclosure is detrimental to the transparency of the reaction enclosure and hence to the production yield of photosynthetic organisms.

To meet all or part of these disadvantages and issues raised above the present invention proposes for this purpose a reaction casing for photosynthetic reactor adapted for the culture of photosynthetic microorganisms, algae in particular, the said reaction casing being designed firstly to float on a body of water and secondly to delimit a biphasic flow pathway for the gas/liquid culture medium between a first and a second opening of the said reaction casing, the said reaction casing being noteworthy in that it comprises two claddings, respectively outer and inner, made at least partly in material transparent to light rays, the inner cladding extending inside the outer cladding so that the said claddings delimit between them an inter-cladding space in fluid connection with the first opening of the reaction casing, in that the outer cladding has an open proximal end and a closed distal end, and in that the inner cladding has one open proximal end in fluid connection with the second opening of the reaction casing and a distal end provided with at least one communication orifice between the inside of the inner cladding and the inter-cladding space.

With this casing, the biphasic flow pathway takes place between the first opening and the second opening, in the inter-cladding space and inside the inner cladding via the communication orifice at the distal end of the inner cladding.

Therefore this casing allows the production of photosynthetic microorganisms, microalgae in particular, by monoclonal culture under controlled conditions, and it can be deployed on the surface of an expanse of water (lake or sea). This casing should therefore contribute towards valorizing these bodies of water which have the largest surface areas and are the least valorized on this planet, for the production of photosynthetic microorganisms.

This casing is intended to take advantage of several intrinsic characteristics of bodies of water, namely:

the thermal inertia related to the high calorific capacity of water which, via exchange with the culture medium through the outer cladding, allows the temperature to be maintained at levels close to the optimum for cultures of photosynthetic microorganisms;

the buoyancy capacity of bodies having a lower density than water which allows the hydrostatic maintaining of the culture volume on the surface along the natural horizontality of bodies of water, thereby avoiding gravitational flows and the formation of undesirable bubbles; and

the transparency of bodies of water when they do not receive any silt.

The body of water can also be used as local water source for the culture but it is desirable to treat this water to remove undesirable microorganisms and certain substances such as excess minerals.

The present casing can be used for the culture of any photosynthetic organisms i.e. any of form of life able to develop by photosynthesis in a suitable, nutrient culture medium, in the presence of sunlight and carbon, in particular in the form of carbon dioxide. This reaction casing adapted to bodies of water allows the following functions to be achieved:

ensuring the confinement of the culture over time by preventing exchanges of matter with the surrounding medium which are liable to entail contaminations and disseminations, and for this purpose to offer resistance to mechanical stresses in particular by currents, wind, and surface agitation;

ensuring photon transfer to deliver sunlight to the microorganisms in culture;

ensuring liquid/gas mass transfer essential for providing carbon and evacuating the reaction oxygen;

ensuring heat transfer to evacuate the calories brought by light rays and to maintain the culture at the right temperature;

lending itself to a reduction in cost prices of the biomass produced, through moderate costs of the means used in this casing.

The casing conforming to the invention allows these results to be obtained and for this purpose provides a particular confinement with two claddings one in the other delimiting a continuous flow pathway for the liquid and gas reaction media, which can be scaled upwards towards large surface areas; the plant production of photosynthetic microorganism production systems is proportional to this surface area in accordance with a factor known as surface productivity whose value is of the order of several tens of grams of dry matter per square meter per day.

Advantageously, at least one of the two claddings, respectively outer and inner, is made in a flexible material adapted to allow folding, inflating, transverse deformation and/or bending of the said cladding. Preferably the two claddings are made in a said flexible material.

By flexible material is meant a material which lends itself to deformation, folding, rolling and bending without rupturing or breaking, such as a flexible or ductile material. Said material is particularly adapted for the casing conforming to the invention since it allows:

folding or rolling of the casing as a whole so that it can be stored in folded or rolled form before being deployed on the body of water by inflation, said inflation being achieved by filling with a gas and/or liquid before these are set in return circulation;

deforming and bending of the inner cladding inside the outer cladding so that the outer wall of the inner cladding is in friction against the inner wall of the outer cladding, thereby cleaning this inner wall of the outer cladding and this outer wall of the inner cladding;

deforming and bending of the outer cladding so that the inner wall of the outer cladding is in friction against the outer wall of the inner cladding thereby cleaning this outer wall of the inner cladding and this inner wall of the outer cladding, so that the outer cladding is in friction against an outer cladding of a neighboring or adjacent reaction casing thereby cleaning the outer walls of these two external claddings;

the manufacturing costs of these casings are reduced with the use of a relatively economical flexible material.

In one particular embodiment, the casing further comprises:

an external connection part on which the proximal end of the outer cladding is hermetically mounted, and on which the first opening of the reaction casing is arranged in fluid connection with the inter-cladding space; and

an internal connection part on which the proximal end of the inner cladding is hermetically mounted, and on which the second opening of the reaction casing is mounted in fluid connection with the proximal end of the inner cladding.

These connection parts, for example in the form of support plates are particularly advantageous for obtaining hermetic or sealed connecting of the two claddings with a return pipe ensuring the casing with fluid connection between the first and second opening of the reaction casing. In addition, these connection parts allows the transmitting of longitudinal tension forces transmitted by the claddings and especially by the outer cladding, transmitting these forces to an embarkation carrying the return pipe in which means are provided for circulating the liquid medium.

According to one characteristic, the proximal end of the inner cladding is mounted in rotation on the internal connection part so that the inner cladding is free to rotate and oscillate inside the outer cladding, thereby promoting cleaning of the walls.

According to another characteristic the internal connection part is mounted inside the external connection part thereby limiting the space taken up by the connection parts with the proximal ends of the claddings being located substantially in the same plane defined by the two parts.

Advantageously the external connection part comprises coupling means for coupling with means for driving the said external connection part in rotation so as to drive the outer cladding in rotation. This driving in rotation of the outer cladding is particularly advantageous for cleaning the outer wall of the outer cladding.

Preferably, the internal connection part is free in rotation in the external connection part so that the rotation of the outer cladding ensures the rotation of the inner cladding by friction between the two claddings. The rotation of the outer cladding is therefore communicated to the inner cladding, promoting movements of the claddings and hence the cleaning of their walls.

In one particular embodiment the inner cladding, shorter than the outer cladding, extends over at least 90% of the length of the outer cladding and preferably over the entire length of the outer cladding less its diameter, so as to optimize the gas/liquid biphasic flow length and hence exchanges between the two phases.

According to one possibility of the invention, the communication orifice provided on the distal end of the inner cladding has a convergence zone to obtain head loss in the gas/liquid culture medium biphasic flow and hence to set up an overpressure which ensures the inflation of the inner cladding over its entire length.

This convergence zone is for example in the form of a reduction in diameter of the inner cladding at the open distal end thereof.

According to another possibility of the invention, the casing further comprises a third cladding in flexible material extending inside the inner cladding to allow the injection or extraction of gas at the distal ends of the two claddings, respectively outer and inner.

If gas is injected into this third cladding, gas return takes place via the inter-cladding space whilst if gas is aspirated from this third cladding the outward flow of the gas takes place via the inner cladding and/or the inter-cladding space. This latter configuration is particularly suited to agitated bodies of water where surface waves tend to displace the injected gas towards the distal end of the two claddings, respectively outer and inner.

In one particular embodiment, the casing further comprises:

removable clamping or ligature means designed to clamp or ligature the two claddings over an intermediate zone located between the respective proximal and distal ends of the two claddings;

at least one intermediate communication orifice between the inside of the inner cladding and the inter-cladding space, the said intermediate communication orifice being provided on the inner cladding between its proximal end and the said intermediate clamping or ligature zone; and

means for closing the said intermediate communication orifice in particular of trap type, the said closure means being mobile between an open position and a closed position.

In this particular embodiment, it is possible to use only a reaction sub-volume, corresponding to the portion of casing between the proximal ends of the claddings and the intermediate clamping or ligature zone, so as to inoculate and place this sub-volume in culture, before placing the complete volume in culture by removing the clamping or ligature means and closing the means for closing the intermediate communication orifice.

In one advantageous configuration, the cross-section of the passageway of the inner cladding is substantially identical to the cross-section of the passageway of the inter-cladding space.

With this configuration, the rate of circulation of the liquid in the inner cladding is allowed to be substantially equivalent to the rate of circulation in the inter-cladding space, which has the advantage of avoiding the onset of decanting zones and other acceleration zones which could cause undesired heterogeneities along the pathway.

Advantageously, the casing further comprises a longitudinal shadow strip partly covering the outer cladding and made in material that is opaque or has strong light absorbance, the shadow strip particularly being added and positioned temporarily on the outer cladding or secured onto the outer cladding.

The use of a said shadow strip is of particular advantage for ensuring temporary protection against luminosity for some cultures which must not be exposed to full light for as long as their concentration is insufficient for protection by self-shadowing.

The invention also relates to a photosynthetic reactor adapted for the culture of photosynthetic microorganisms, algae in particular comprising:

at least one reaction casing conforming to the invention;

at least one return pipe ensuring fluid connection between the first and second openings of the said reaction casing;

at least one means for setting in circulation arranged in the said return pipe and designed to set in circulation the liquid culture medium in the return pipe and in the reaction casing;

at least one liquid injection means arranged in the said return pipe and designed to allow the injection of liquid into the reaction casing;

at least one means for injecting gas arranged in the said return pipe and designed to allow the injection of gas into the reaction casing; and

at least one gas escape means arranged in the said return pipe and designed to allow the escape of injected gas from the reaction casing.

This reactor may evidently comprise several reaction casings with circulation means common to all these casings.

With a reactor according to the invention the liquid culture medium and the gas circulate simultaneously in contact with one another along the biphasic flow pathway that is substantially horizontal since the reaction casing and hence the claddings delimiting this pathway float on the surface of the water which is mainly horizontal (to within variations caused by wind, waves, surface movements, etc.), and exchange some components along their common pathway. The exchanges between the liquid culture medium and the gas are proportional to the length of the claddings, which allows the envisaging of extensive scale-up.

The reactor and the reaction casing conforming to the invention are therefore especially designed to increase the efficacy of gas/liquid transfer and to reduce mechanical stresses inflicted on the cultivated organisms so as to extend production to fragile species.

In addition, the reactor and the reaction casing conforming to the invention allow limiting of the formation of small-diameter bubbles and thereby reduce the development of oxygen-consuming heterotrophic microorganisms. With the reaction casing of the invention, gas/liquid transfer no longer occurs inside a vertical bubble column but along the substantially horizontal flow pathway in which the flow follows a pattern of horizontal biphasic type, in particular of stratified flow type, or slug or elongated bubble flow.

Contrary to the principle mentioned above whereby the reaction only takes place in the liquid phase, the Applicant started out from the principle according to which the gas is an integral part of the reaction and must be allowed to enter the reaction volume in the same way as the liquid. By giving priority to flow patterns of horizontal biphasic type (stratified, slug or elongated bubble) the exchange surface between the gas and the liquid is extended to the entirety of the pathway in the reaction casing with bubble production that is distinctly less abundant than is the case in some prior art reactors, thereby reducing the deleterious effect of these bubbles that has been observed.

In addition, in the reactor of the invention, the setting in circulation of the liquid culture medium is ensured by one or more circulation means generating reduced shear and centrifugal forces. The circulating function is separate from the gas-liquid exchange function, unlike the case with gas-lift reactors.

The invention also concerns a method for the culture of photosynthetic microorganisms, algae in particular, using a reactor conforming to the invention and comprising the following steps:

injecting a liquid culture medium into the reaction casing at a controlled flow rate using the liquid injection means;

injecting a gas into the reaction casing at a controlled flow rate using the gas injection means;

setting the liquid culture medium in circulation using the circulation means;

controlling the circulation means and gas injection means to set up inside the reaction casing a biphasic flow of the gas/liquid culture medium that is of stratified flow type, or slug flow or elongated bubble flow.

According to one characteristic, the control step comprises a step to control the rate of circulation of the liquid in the reaction casing at between 0.1 and 1.0 m/s, preferably between 0.1 and 0.5 m/s.

According to one characteristic, the control step comprises a step to control the rate of circulation of the liquid in the reaction casing so that the rate of circulation in the inner cladding is substantially equivalent to the rate of circulation in the inter-cladding space; these circulation rates each possibly being between about 0.1 and 1.0 m/s, and preferably between 0.1 and 0.5 m/s.

This characteristic is advantageous to avoid the onset of decanting zones and other acceleration zones which may create undesirable heterogeneities along the pathway. This control is preferably obtained with a configuration such that the cross-section of the passageway of the inner cladding is substantially identical to the cross-section of the passageway of the inter-cladding space.

According to another characteristic, the circulation means comprise a propeller driven in rotation by a motor, and the speed of rotation of the propeller is less than about 1000 rotations per minute, preferably less than about 100 rotations per minute.

According to one characteristic the injection of the liquid culture medium and of the gas into the reaction casing is performed to inflate and deploy the casing on the surface of the body of water.

The invention also relates to a method for manufacturing a reaction casing conforming to the invention, comprising the following steps:

fabricating the inner cladding, in particular by extruding a plastic material and blowing the extruded plastic;

fabricating an outer sheet in plastic material in particular by calendaring;

wrapping the inner cladding with the outer sheet as far as the junction of two opposite edges; and

welding the outer sheet along its two opposite edges joined at the wrapping stage, so as to form the outer cladding surrounding the inner cladding.

These steps can be completed by a rolling or folding step of the inner and outer claddings thus formed.

The manufacturing method conforming to the invention is particularly economical and rapid.

### BRIEF DESCRIPTION OF THE DRAWINGS

Other characteristics and advantages of the present invention will become apparent on reading the detailed description given below of several examples of non-limiting embodiments, given with reference to the appended figures in which:

FIG. 1 is a schematic cross-sectional view of a casing conforming to the invention;

FIG. 2a is a schematic rear view of a casing conforming to the invention, illustrating a connection part common to the claddings of the casing;

FIG. 2b is a partial longitudinal section view along axis II-II of the casing illustrated in FIG. 2a, showing the proximal ends of the claddings;

FIGS. 3a and 3b are schematic longitudinal section views of two casings conforming to the invention, illustrating two variants of embodiment of a communication orifice provided at the distal end of the inner cladding;

FIG. 4 is a schematic illustration giving an overhead and profile view of a reactor conforming to the invention comprising several casings moored only on one side on a floating embarkation;

FIG. 5 is a schematic overhead view of another reactor conforming to the invention comprising several casings moored on both sides onto an embarkation and a beam and swivel system respectively;

FIG. 6a is a partial schematic profile view of a reactor conforming to the invention comprising a return pipe integrating means for circulating the liquid medium according to a first embodiment;

FIG. 6b is a cross-sectional view along axis VI-VI of the right part of the reactor illustrated in FIG. 6a;

FIG. 7a is a partial schematic profile view of a reactor conforming to the invention comprising a return pipe integrating means for circulating the liquid medium according to a second embodiment;

FIG. 7b is a cross-sectional view along axis VII-VII of the right part of the reactor illustrated in FIG. 7a;

FIG. 7c is a cross-sectional view along axis VII-VII of the left part of the reactor illustrated in FIG. 7a;

FIG. 7d is an overhead view of the reactor illustrated in FIG. 7a;

FIG. 8a is a partial schematic profile view of a reactor conforming to the invention comprising a return pipe integrating means for circulating the liquid medium according to a third embodiment;

FIG. 8b is a rear view of the reactor illustrated in FIG. 8a;

FIG. 9a is a partial schematic profile view of a reactor conforming to the invention comprising a return pipe integrating means for circulating the liquid medium according to a fourth embodiment;

FIG. 9b is a rear view of the reactor illustrated in FIG. 9a;

FIGS. 10a and 10b are schematic longitudinal section views of a casing conforming to the invention, respectively illustrating a clamping step of the casing so as only to place a sub-volume of the reaction casing in culture, and a step to remove the clamping means so as to place in culture the entire volume of the casing;

FIGS. 11a and 11b are schematic profile views of a reactor conforming to the invention comprising a casing moored on one side onto a floating embarkation, respectively illustrating a step to place the casing in place on the return pipe and a step to deploy the casing over the body of water by inflating or filling;

FIG. 12a is a schematic cross-sectional view of two reactors conforming to the invention connected together via link lines;

FIG. 12b is a schematic partial overhead view of the two reactors illustrated in FIG. 12a;

FIG. 13a is a schematic partial overhead view of a reactor comprising several reaction casings connected to a return pipe integrating circulation means common to all the casings;

FIG. 13b is a longitudinal section view along axis XIII-XIII of the reactor shown in FIG. 13a;

FIG. 14 is a schematic view illustrating a system for manufacturing a casing conforming to the invention;

FIG. 15 is a schematic perspective view of reactors conforming to the invention comprising casings moored onto an offshore platform.

### DETAILED DESCRIPTION

The description of a reaction casing 1 conforming to the invention for a photosynthetic reactor 2, or photobioreactor, is given with reference to

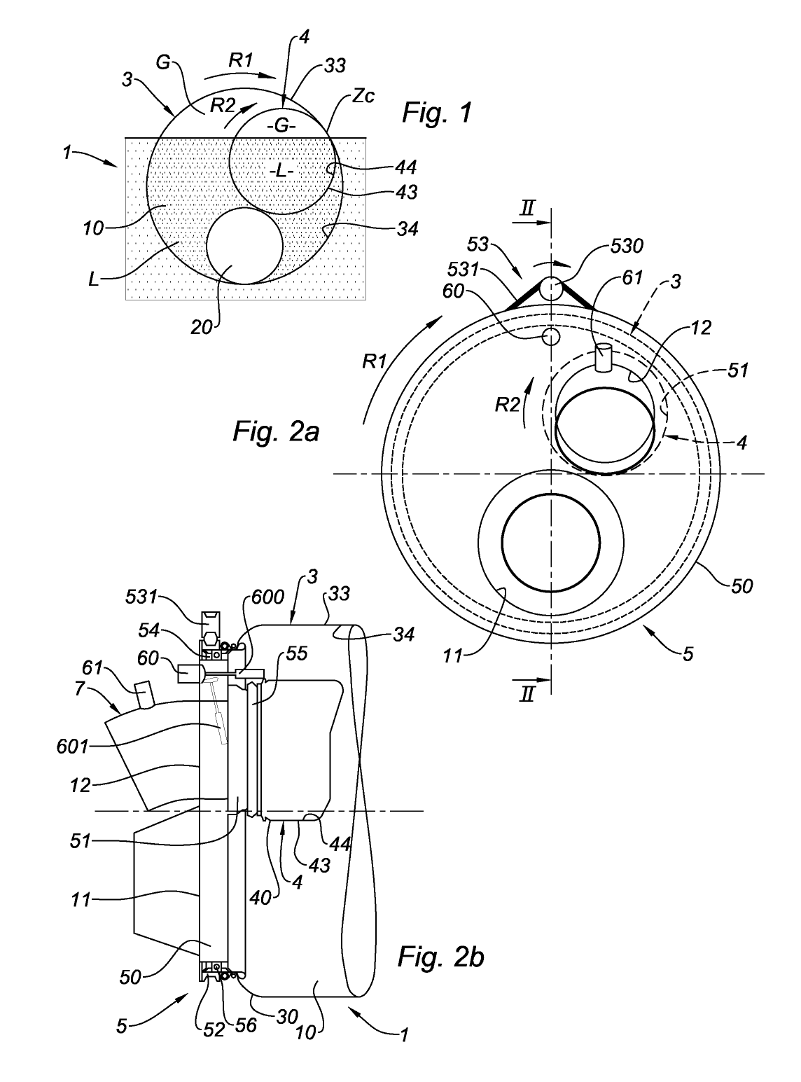

FIGS. 1 to 3; this casing 1 being adapted for the culture of photosynthetic microorganisms, algae in particular and in particular for the culture of photosynthetic microorganisms fragile to mechanical stresses.

The casing 1 is designed firstly to float on the body of water and secondly to delimit a biphasic flow pathway for the gas/liquid culture medium between a first opening 11 and a second opening 12 of the casing 1. For this purpose the casing 1 comprises:

an outer cladding 3 made at least partly of a flexible material transparent to light, in which the outer cladding 3 lies lengthwise in particular of general tubular shape and has an open proximal end 30 and a closed distal end 31 in other words ending in a cul-de-sac;

an inner cladding 4 made at least partly of a flexible material transparent to light rays, in which the outer cladding 3 lies lengthwise generally of tubular shape and extends inside the outer cladding 3 so that these two claddings 3, 4 delimit between them an inter-cladding space 10 and has one open proximal end 40 and a distal end 41 provided with at least one communication orifice 42 between the inside of the inner cladding 4 and the inter-cladding space 10;

an external connection part 50 on which the proximal end 30 of the outer cladding 3 is hermetically mounted, and on which the first opening 11 of the casing 1 is arranged which is in fluid connection with the inter-cladding space 10; and

an internal connection part 51 on which the proximal end 40 of the inner cladding 4 is mounted, and on which the second opening 12 of the casing 1 is arranged which is in fluid connection with the proximal end 40 of the inner cladding 4.

The outer cladding 3 has a length of several tens of meters and a decimetric so-called outer diameter De of between 5 and 50 cm for example.

The inner cladding 4 has:

a length substantially equivalent to the length of the outer cladding 3, for example a length of more than 90% of the length of the outer cladding 3 and preferably equal to the length of the outer cladding less its diameter; and

a so-called inner diameter Di substantially smaller than the diameter of the outer cladding 3.

The inner cladding 4 is therefore freely deployed inside the outer cladding 3 over about its entire length.

The Applicant has observed that it is preferable for the rates of circulation in the inner cladding 4 and in the inter-cladding space 10 should be as close as possible to each other, even identical to avoid the onset of some decanting zones and other acceleration zones which create undesirable heterogeneities along the pathway.

For this purpose, it is necessary and it suffices that the passageway cross-sections of the inner cladding 4 and of the inter-cladding space 10 are identical, which can translate as follows with respect to tubular claddings 3, 4:

∩(De²-Di²)/4=∩ Di²/4 , and hence the following relation R1 is obtained: De=2^{1/2}.Di.

Expediently, the rates of circulation in the inner cladding 4 and in the inter-cladding space 10 are between 0.1 and 0.5 m/s.

The two claddings 3, 4 are made in a flexible material, in other words in a material adapted to allow the folding, inflating, transverse deformation and/or bending of the claddings 3, 4.

Regarding the resistance of the claddings 3, 4, the membrane of the outer cladding 3 must be able to withstand the traction force due to movement of the mass of the body of water, that of air being negligible. This force is substantially equivalent to the drag force of a ship of same length on wetted surface area for one same relative speed. This approach for calculating the traction forces to which the claddings 3, 4 are subjected and which are transmitted to a mooring support (floating embarkation E described below, quayside or bank) via the connection parts 50, 51, gives an underestimate however since it does not take jolting into account. A safety margin must therefore be provided bearing in mind that only the outer cladding 3 transmits these forces.

The Applicant has therefore drawn up a list of plastic materials which can be used to fabricate the claddings 3, 4, which notably comprises polyethylene, polypropylene, the polyamides (Nylon, Rylsan), polytetrafluoroethyenes (PTFE), either in membrane form or in the form of woven fibers or in the form of calendared or coated fabric composites. This list is evidently not limiting and may be completed in particular by new transparent materials appearing on the market.

The membranes or films made in these plastic materials have tensile strengths of several tens of kilograms per linear meter, which is of the order of a hawser of rope for marine use of small diameter. With such materials the outer cladding 3 can therefore withstand traction forces induced by wind. It is therefore advantageous to take care that the mooring support such as the floating embarkation also transmits as little jolting as possible to the casing 1 and to its claddings 3, 4.

As illustrated in FIGS. 2a and 2b, the internal connection part 51 is mounted inside the outer connection part 50 so that these two connection parts 50, 51 together form a connection or securing plate 5 intended to allow the connecting of the claddings 3, 4 with A return pipe 7 described below.

The external connection part 50 comprises coupling means 42 with the means 53 for driving in rotation the external connection part 50 so as to drive the outer cladding 3 in rotation. Therefore the proximal end 30 of the outer cladding 3 is hermetically associated with a rotating fixture namely the external connection part 50. The speed of rotation implemented by the driving means 53 is of the order of a few rotations per day.

In the embodiment illustrated in FIGS. 2a and 2b the coupling means 52 are in the form of a pulley arranged on the cylindrical periphery of the external connection part 50, and the driving means 53 comprise a rotary motor provided with a rotating output shaft 530 on which a belt or chain 531 is mounted meshing with the pulley 52.

The internal connection part 51 included in the connection plate 5 is freely rotatable in the external connection part 50 so that the rotation of the outer cladding 3 by the driving means 53 ensures the rotation of the inner cladding 4 by friction between the two claddings 3, 4. Therefore the proximal end 40 of the inner cladding 4 is mounted in rotation on the plate 5. In other words the proximal end 40 of the inner cladding 4 is associated hermetically with a rotating fixture namely the internal connection part 51.

The connection plate 5 is intended to transmit the longitudinal tensile forces transmitted by the claddings 3, 4 and especially the outer cladding 3, transmitting these forces to the mooring embarkation E.

This connection plate 5 is partly immersed in the body of water and has a uniform or alternating rotating movement so that it ensures:

the setting in rotation or oscillation of the claddings 3, 4 with an angle amplitude equal to or more than 360°, i.e. a complete rotation, this rotation being illustrated by the arrow R1 in FIGS. 1 and 2a;

the free rotation of the inner cladding 4 inside the outer cladding 3, this rotation being illustrated by the arrow R2 in FIGS. 1 and 2a; and

maintaining of the integrity/seal of the confinement formed by the casing 1 allowing these rotational movements to take place hermetically.

To obtain this hermetic securing and therefore to avoid leakages, seals 54 equipped with an antifriction and sealing device are provided between the attachment of the connection plate 5 and the outer cladding 3; since the inner volume of the casing 1 is pressurized, any leaks flow outwardly which limits risks of contamination. Seals 55 equipped with an antifriction and sealing device are also provided between the mounting of the connection plate 5 and the inner cladding 4 although the consequence of any leak at this level would not be serious.

To reduce the rotational driving force of the connection plate 5, the plate 5 is provided with ball bearings 56 underneath the pulley 52.

The rotation of the outer cladding 3 is communicated to the inner cladding 4 whose internal connection part 51 is free in rotation as described above in the plate 5 by friction thereof.

As can be seen in FIGS. 2a and 2b, the securing of the inner cladding 4 onto the plate 5 is slightly offset upwardly:

to allow installation of the gas outlet, also called the gas escape means 60 in the upper part; and also

to promote the contact between the two claddings 3, 4, this contact ensuring the driving in rotation or in oscillation, of the inner cladding 4 by the outer cladding 3.

Expediently, and as can be seen in FIG. 2b, the orifice of the gas escape means 60 i.e. the gas outlet is equipped with a shut-off with float 600 intended to prevent passing of the liquid.

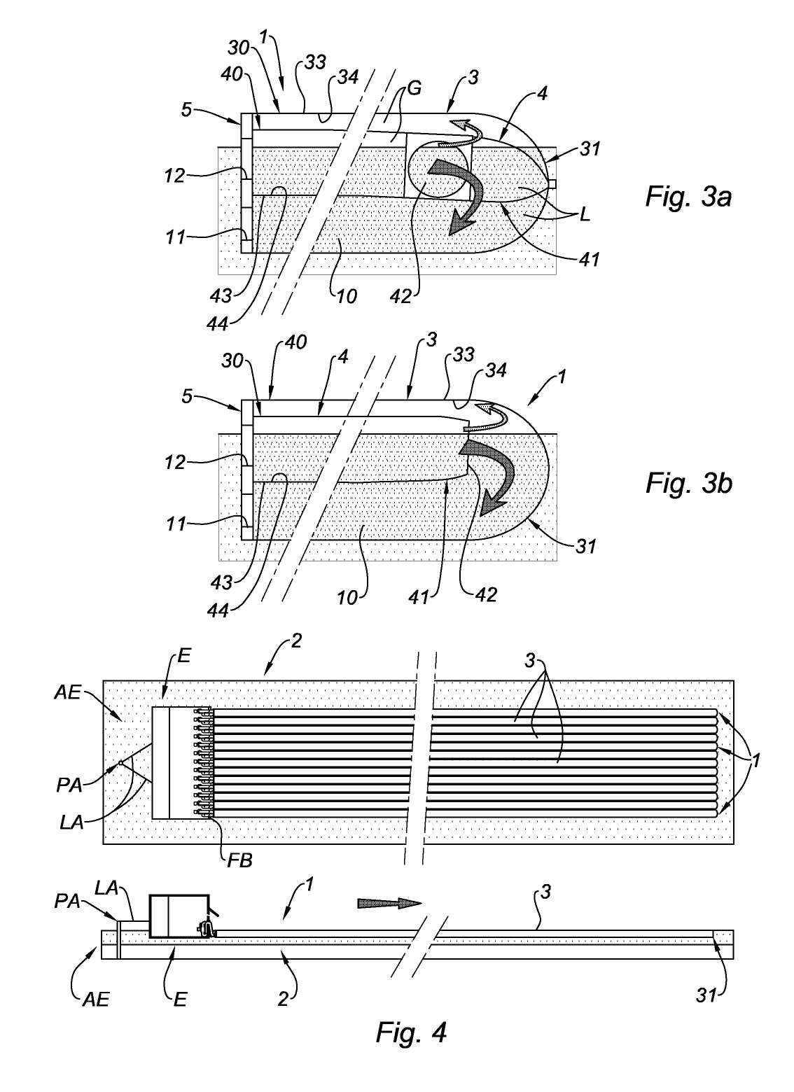

FIGS. 3a and 3b illustrates two casings in two separate embodiments of the communication orifice 42. In the embodiment illustrated in FIG. 3a, the communication orifice 42 is arranged on the side wall of the inner cladding 4, in the form of a window optionally provided with reinforcements on its edges, where the distal end 41 of the inner cladding 4 is secured in particular by welding or clamping or a by common ligature onto the distal end 31 of the outer cladding 3. In the embodiment illustrated in FIG. 3b the communication orifice 42 is arranged at the end of the distal end 41 of the inner cladding 4 so that the distal end 41 is said to be open and free.

The cleaning of the transparent walls of the claddings 3,4 is one of the main advantages of the casing 1 conforming to the invention. The purpose of this cleaning is to prevent the development of biofilms on their respective walls to avoid a reduction in transmitted light which is detrimental since it slows the reaction.

To perform this cleaning several functions are used:

the relative movements and the friction of adjacent outer claddings 3 against each other allows the cleaning of their respective outer walls 33, these relative movement being obtained in particular by deformation and bending of the flexible outer claddings 3 under the action of the agitation of the body of water induced in particular by the wind (lapping);

the rotation of the outer cladding 3 over at least one rotation (angle amplitude equal to or more than 360°), allows the extending of the above-mentioned cleaning of their outer walls 33 to their entire periphery;

the relative movements between the two claddings 3, 4, caused by rotation of the outer cladding 3, associated with the action of the agitation of the body of water and the flexibility of their materials, leads to friction of the inner cladding 4 against the outer cladding 3 which allows cleaning of the inner wall 34 of the outer cladding 3 and the cleaning of the outer walls 43 of the inner cladding 4;

the rotation of the outer cladding 3 over at least one rotation (angle amplitude equal to more than 360°, allows the above-mentioned cleaning of the inner wall 34 of the outer cladding 3 and the cleaning of outer walls 43 of the inner cladding 4 to be extended to their entire periphery.

Therefore the contact between the two claddings 3, 4 of one same casing 1 has the effect of avoiding the development of a biofilm and ensuring the cleaning both of the inner wall 34 of the outer cladding 3 and the outer wall 43 of the inner cladding 4. The rotation, or oscillation, of angle amplitude equal to or more than 360°, is intended to be such that this cleaning is performed over the full periphery of the claddings.

As illustrated in particular in FIGS. 1 and 3, the liquid culture medium L and the gas G circulate simultaneously in contact with one another and exchange matter through a gas-liquid interface along the reaction pathway from the inside of the inner cladding 4 as far as the inter-cladding space 10, or conversely. The shape of this inter-cladding space 10 depends in particular on the relative position of the two claddings 3, 4 and on the flow pattern which itself is governed by the gas and liquid flow rates.

As illustrated in particular in FIGS. 1 and 3, since the density of the constituent material of the claddings 3, 4 differs little from that of water, the mean levels of the gas-liquid interface in the inter-cladding space 10 and in the inner cladding 4 are substantially identical to that of the body of water; these levels determining the inner and outer waterlines.

As can be seen in FIG. 1, the contact zone ZC between the inner cladding 4 and the outer cladding 3 is located above the waterline. As a result, the inter-cladding space 10 has an asymmetric section of closed croissant shape as shown in FIG. 1. The inner cladding 4 tends to remain in contact with the outer cladding 3 under capillary forces applied by the liquid film lying in the contact zone ZC between the two claddings 3, 4 and the inter-cladding space 10. The liquid film of the contact zone ZC which is of narrow thickness is maintained by capillarity from the culture medium L circulating in the inter-cladding space 10.

The connecting or contact force between the two claddings 3, 4 is weak and can be locally and temporarily cancelled by the movements of the body of water and deformation of the claddings 3, 4 caused by this agitation. This weak contact and the movements of the body of water mean that the contact is accompanied by sliding and friction which prevent the development of a biofilm and promote the cleaning of the walls 43 and 34 concerned by the contact, namely the inner wall 34 of the outer cladding 3 and the cleaning of the outer walls 43 of the inner cladding 4.

As described above, the rotation of the outer cladding 3 is transmitted to the inner cladding 4 by friction in the contact zone ZC, despite the sliding which deteriorates the quality of driving. A certain stiffness of the inner cladding 4 is necessary to prevent pleating thereof and to generalize the cleaning effect to the walls 43 and 34. To avoid the consolidation of the biofilm between two contacts it is sufficient for example that the rate of rotation is a few times a day as suggested above.

The two claddings 3, 4 channel the pathway of the gas and liquid culture medium in a biphasic gas G/liquid culture medium L flow pathway (or reaction pathway) forming an outward and return journey between the openings 11, 12 of the casing 1, in the inner cladding 4 and in the inter-cladding space 10 separating the inner cladding 4 from the outer cladding 3.

The circulation of gas in the claddings 3, 4 sets up positive buoyancy distributed homogeneously over the length of the claddings 3, 4 holding them on the surface. Horizontality is ensured naturally by buoyancy of the body of water and by the circulation of gas. The agitation of the body of water under the effect of wind can translate as longitudinal deformations of the outer cladding 3, which can be transmitted to the inner cladding 4, the flexibility of suppleness of the claddings 3, 4 allowing return to the initial shapes.

Preferably the onset of pleating must be avoided which would substantially reduce the reaction volume. Said pleats could cause jolting and other hammering in the event of agitation of the body of water, which would cause sudden tensions in the flexible material of the claddings 3, 4 possibly causing tearing of the claddings 3, 4 under consideration. For this purpose, the outer cladding 3 must be taut permanently by means of inflation overpressure; controlling of this overpressure must maintain the outer cladding at a nominal level compatible with the agitation of the body of water and the proper functioning of the assembly.

Therefore a method for the culture of photosynthetic microorganisms using a said casing 1 comprises a pressurizing step of the outer cladding comprising setting up inflation overpressure in this outer cladding 3.

The inflation overpressure of the outer cladding 3, as described above, determines its stiffness in other words its resistance to deformation related to agitation of the body of water and the influence of the latter on the internal biphasic flow of gas G and liquid L; this overpressure being equal to the sum of the gas and liquid injection pressures into the volume of the outer cladding 3.

Controlling of the inflation overpressure is also intended to detect leaks. The outlet of the excess gas volume is the escape means 60 described above and provided with an orifice arranged in the upper part of the plate 5 as shown in FIG. 2b. The gas is channeled for example as far as a filter which allows back-contamination of the reactor to be avoided, before being released into the atmosphere or recycled. Controlling of the outlet flow of the gas can be performed using gas flow control means 601 such as a needle valve in order to adjust the headspace (level of the liquid or gas/liquid interface) in the claddings 3, 4.

FIGS. 4 to 13 illustrates photosynthetic reactors 2 conforming to the invention and adapted for the culture of photosynthetic microorganisms, algae in particular. Each reactor 2 comprises:

at least one casing 1 conforming to the invention;

at least one return pipe 7 (which can be seen in particular in FIGS. 6, 7, 8, 9, 12 and 13) ensuring fluid connection between the first 11 and the second 12 opening of the casing 1, the said openings 11, 12 being arranged in the connection plate 5 on which the said return pipe 7 is sealingly connected;

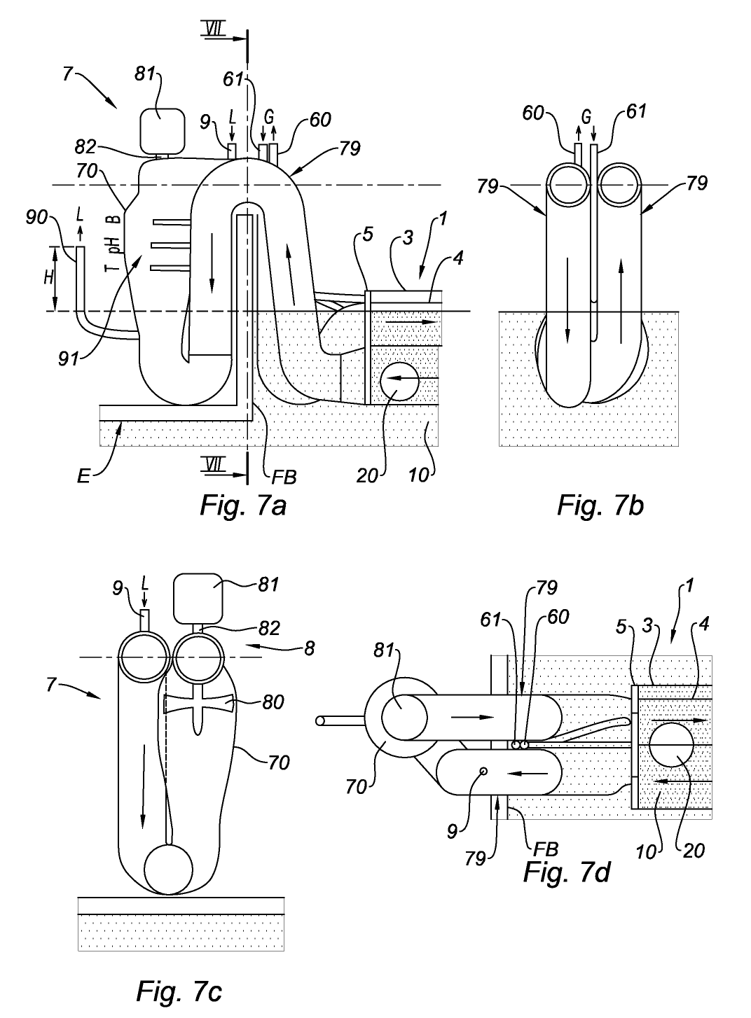

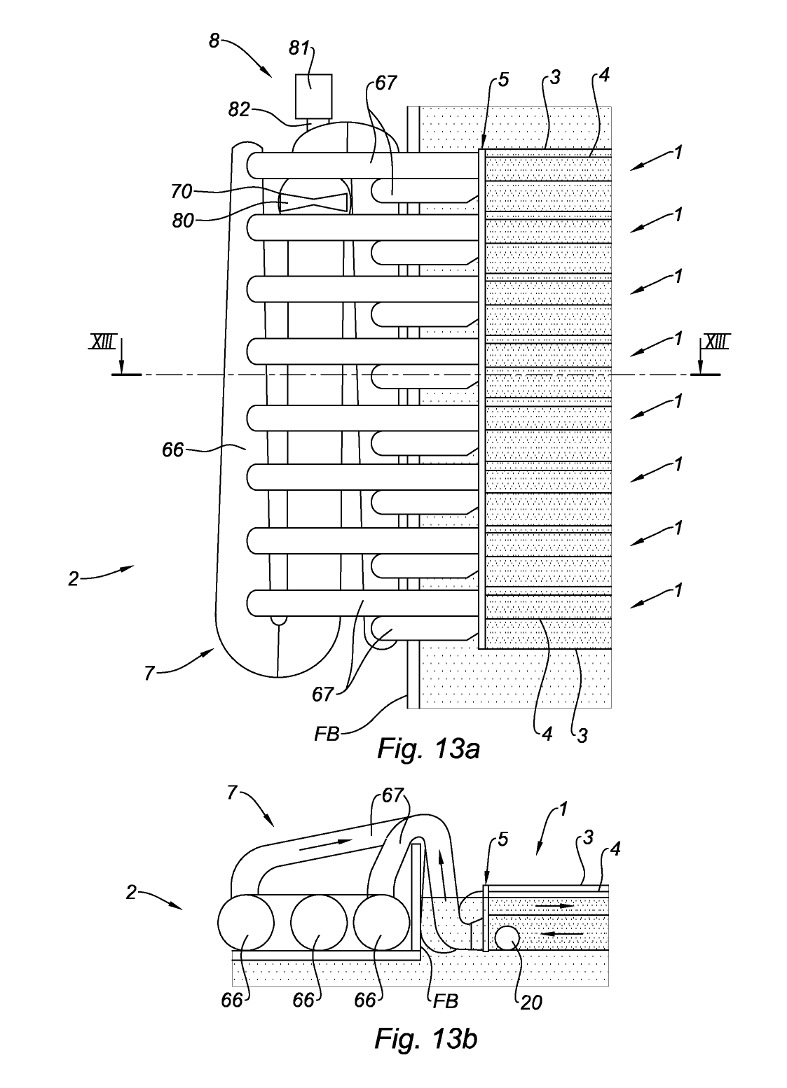

at least one circulation means 8 (shown in FIGS. 7c and 13a) arranged in the return pipe 7 and designed to circulate the liquid culture medium L in the return pipe 7 and in the casing 1;

at least one liquid injection means 9 arranged in the return pipe 7 and designed to inject liquid L into the casing 1;

at least one gas injection means 61 arranged in the return pipe 7 and designed to allow the injection of gas G into the casing 1; and

at least one gas escape means 60 arranged in the return pipe and designed to allow the escape of gas G injected into the casing 1.

The reactor 2 may comprise two separate liquid injection means 9 allowing injection of the liquid culture medium and of inoculum respectively into the reactor 2. These injection means are in the form of injection ports allowing connection to a source with control of asepsis.

The reactor 2 may comprise:

one or more sensors 91 arranged n the return pipe 7 and adapted to provide the necessary signals for controlling the reaction, in particular signals representing physical, chemical or biological parameters for the quality of the culture such as temperature, pH, level of dissolved oxygen and turbidity of the liquid medium, etc. these controls being used in particular to adjust the injections of gas and liquid into the reactor 2;

means for controlling the sterility of the gas and liquid media entering and leaving the space confined by the reactor 2;

regulation loops intended to regulate the main nutrient supplies for the culture, particularly sterile medium additions via the concentration of dry matter, the pH by the injection of CO₂.

The return pipe 7 ensures the closing of the looped fluid pathway between the first 11 and second 12 openings of the casing 1. The return pipe 7 is made in a material non-transparent to sunlight and/or can be arranged away from light inside an enclosure or a closed embarkation E as can be seen in particular in FIGS. 4 and 11.

The function of the circulation means 8, inserted between the return pipe(s) 7 is to circulate the liquid culture medium inside the casing 1. Preferably the circulation means 8 are chosen to generate reduced shear and centrifugal forces. It is possible however to use all types of pumping means and in particular centrifugal pumps without departing from the scope of the invention.

Preferably the circulating function is separate from the gas-liquid mass exchange which is ensured via their interface in the claddings 3, 4 of the casing 1 and which is applied over their entire length. However, one particular embodiment of the reactor according to the invention that is not illustrated may comprise the application of a solution of gas-lift type described above which ensures both pumping and the exchange of gas-liquid mass without departing from the scope of the invention.

The reactor 2 is particularly advantageous in that it applies the principle according to which the gas is an integral part of the reaction and must be allowed into the reaction volume in the same way as the liquid by using a horizontal biphasic flow in the claddings 3, 4 of the casing 1 floating on the body of water; these two flexible claddings 3, 4 included one in the other creating an outward and return pathway for the gas and the liquid.

With said casing and hence with said reactor 2 four possible configurations of biphasic pathway can be envisaged:

a first pathway: co-current circulation with the liquid L and the gas G entering the casing 1 via the second opening 12 and leaving the casing 1 by the first opening 11, so that the liquid L and the gas G perform an outward journey inside the inner cladding 4 and a return journey in the inter-cladding space 10;

a second pathway: co-current circulation with the liquid L and the gas G entering the casing 1 via the first opening 11 and leaving the casing 1 via the second opening 12, so that the liquid L and the gas G perform an outward journey in the inter-cladding space 10 and a return journey in the inner cladding 4;

a third pathway: counter-current circulation with the liquid L entering the casing 1 via the second opening 12 and leaving the casing 1 via the first opening 11, and with the gas G entering the casing via the first opening 11 and leaving the casing 1 via the second opening 12, so that the liquid L performs an outward journey in the inner cladding 4 and a return journey in the inter-cladding space 10 and the gas performs an outward journey in the inter-cladding space 10 and a return journey in the inner cladding 4; and

a fourth pathway: counter-current circulation with the liquid L entering the casing 1 via the first opening 11 and leaving the casing 1 via the second opening 12, and the gas G entering the casing 1 via the second opening 12 and leaving the casing 1 via the first opening 11 so that the liquid L performs an outward journey in the inter-cladding space 10 and a return journey in the inner cladding 4, and the gas G performs an outward journey in the inner cladding 4 and a return journey in the inter-cladding space 10.

With regard to the first pathway, the gas G and the liquid culture medium L are injected into the inner cladding 4 at the second opening 12 arranged in the plate 5 and escape from this inner cladding 4 via the communication orifice 42 provided at the distal end 41, then return to the plate 5 in the inter-cladding space 10.

With regard to the fourth pathway, the gas G is injected into the inner cladding 4 whilst the liquid culture medium L is injected into the inter-cladding space 10; the return of the gas G occurring in the inter-cladding space 10 and the return of the liquid L in the inner cladding 4.

In all cases, the liquid L on its return is taken up by a pump located in the return pipes 7 to be re-introduced into the same outward journey, whilst the gas G must be released into the atmosphere or recycled via the gas escape means 60.

Preferably, the communication orifice 42 provided on the distal end 41 of the inner cladding 4 has a convergence zone to provide a head loss in the biphasic flow of gas/ liquid culture medium. In the embodiment illustrated in

FIG. 3b, the open distal end 41 of the inner cladding 4 has a narrowed diameter to create this original head loss; the overpressure created by this convergence or restriction zone ensuring the inflation of the inner cladding 4 over its entire length.

Optionally, a third cladding (not illustrated) also made in a flexible material transparent to light rays, whose wall is sufficiently stiff so that it can undergo a pressure drop without collapsing and of small diameter (compared with the diameter of the inner cladding 4) is located inside the inner cladding 4 to allow the injection or extraction of gas at the distal end 31, 41 of the two claddings 3, 4.

As can be seen in FIG. 1, the circulation of gas G and of liquid L, in counter-current or co-current direction, occurs along a substantially rectilinear horizontal pathway in which the gas G gathers in a volume located in the upper or top part of the cavity delimited by the two claddings 3, 4.

This creates an interface between the gas G and the liquid L which is the seat of transfers related to the photosynthesis reaction. The longitudinal shape of this interface and in particular its continuous or discontinuous nature; characterize what is called the flow pattern. Without considering the influence of the agitation of the body of water, the flow systems in the inner cladding 4 are substantially the same as in a horizontal pipe of circular cross-section. These flow patterns have been described under the names of stratified flow, slug flow or elongated bubble flow. The flow patterns in the inter-cladding space 10 are slightly perturbed by the presence of the inner cladding 4 but will have substantially identical characteristics with respect to the mass transfer.

With regard to biphasic flows in horizontal conduits, work has evidenced several flow systems depending on the conditions of velocity, diameter, temperature, type and pressure of the circulating fluids, namely in particular:

dispersed bubble flow, of Mandhane AD typology; and

elongated bubble flow, of Mandhane I typology;

stratified flow, with wave stratified flow and smooth stratified flow of Mandhane SS and SW typology;

slug flow, of Mandhane I typology;

annular mist flow, of Mandhane AD typology.

In the present invention, the flow patterns given priority therefore lie at the transition SS/I transition in Mandhane typology, i.e. between the stratified flow and the slug or elongated bubble flow. With the stratified flow pattern, the gas/liquid interface is formed by the free surface whose width varies with the level of the liquid in the claddings 3, 4. With the slug or elongated bubble flow pattern the gas/liquid interface is formed by the floor and the ceiling of the slug or elongated bubble.

Since mass transfers are proportion to the path length, the effect of this length on performance levels of the reaction is reduced, which allows the envisaging of major scale-up. The cladding 3, 4 can have lengths of several hundred meters.

In the particular case of the first pathway described above, the gas G and the liquid L are injected simultaneously into the inner cladding 4. To avoid reflux of the gas G in counter-current to the liquid L the gas G is preferably injected at an injection point 61 located downstream of a low point of the ceiling of the return pipe 7 which acts as backstop, as illustrated in FIGS. 6, 7, 8 and 9. The separation of the liquid L and the gas G is completed by the positioning of the first opening 11 which forms the outlet of the inter-cladding space 10 in the lower part of the plate 5. This separation is essential so as not to deteriorate the performance of the circulation means 8.

The outlet of the volume of liquid L in excess in the reactor normally occurs via an overspill formed by an outlet pipe 90 (visible in FIGS. 6a, 7a, 8, 9 and 12) in communication with the return pipe 7. The free end of this outlet pipe 90 is set at a height H which can be adjusted in relation to the body of water; this height H which can be seen in FIGS. 6a and 7a determining the inflation overpressure of the outer cladding 3 and able to be adjusted to stabilize the inner flow in relation to the agitation of the body of water. This height H is of the order of a few centimeters and may exceed several decimeters for claddings 3, 4 of great length.

The communication point of this outlet pipe 90 of the liquid L with the return pipe 7 is placed as far away as possible and preferably upstream of the injection point(s) 9 of sterile liquid medium as illustrated in FIGS. 6a and 7a, the arrows in the figures illustrating the direction of circulation of the liquid medium L. It is at the outlet of this outlet pipe 90 that the culture is harvested and short-circuiting must be avoided which would dilute the harvest.

This outlet pipe 90 forming an overspill forms a rupture in the confinement of the liquid culture medium L. To avoid back-contaminations of the culture in progress the outlet pipe 90 can usefully have a length of several meters and be held sterile by periodical cleaning.

As can be seen in FIGS. 4, 5 and 11, the return pipe 7, the circulation means 8 and the plate 5 are fixed to an embarkation E resting on the water; this embarkation E may be of floating type and form a floating embarkation or barge, or it may be of pontoon type with beams or timbers planted into the bottom of the body of water.

This embarkation E may comprise a closed space or technical space in which the return pipe 7 and the circulation means 8 are arranged protected against bad weather, and a wall or freeboard FB on which the claddings 3, 4 are attached to this embarkation E via the plate 5; since the distal ends 31, 41 of these claddings 3, 4 are left free allowing them to align with the direction of relative movement of the mass of water supporting the assembly, this has the effect of reducing drag forces related to the movement of this supporting mass of water. A said production assembly optimizes the use of common means such as the embarkation E and the on-board functionalities.

In the embodiments illustrated in FIGS. 4 and 11 the embarkation E is floating and anchored to the bottom of the body of water which here is fairly shallow by a single mooring AE of the embarkation E, so that the outer claddings 3 of the casings 1 may align themselves from this embarkation E with the field of the current induced by the wind, oscillating freely about the single mooring AE. This mooring AE comprises a mooring point PA particularly in the form of a beam or rod planted vertically into the bottom of the body of water, and mooring lines LA connecting the embarkation E to the mooring point PA and leaving the said embarkation E free in rotation about the mooring point PA. If the mooring AE uses a single mooring point PA as is the case in the embodiments illustrated in FIGS. 4 and 11, sufficient space must be provided so that the assembly is able to move freely around this point under the influence of currents, in particular wind-induced currents.

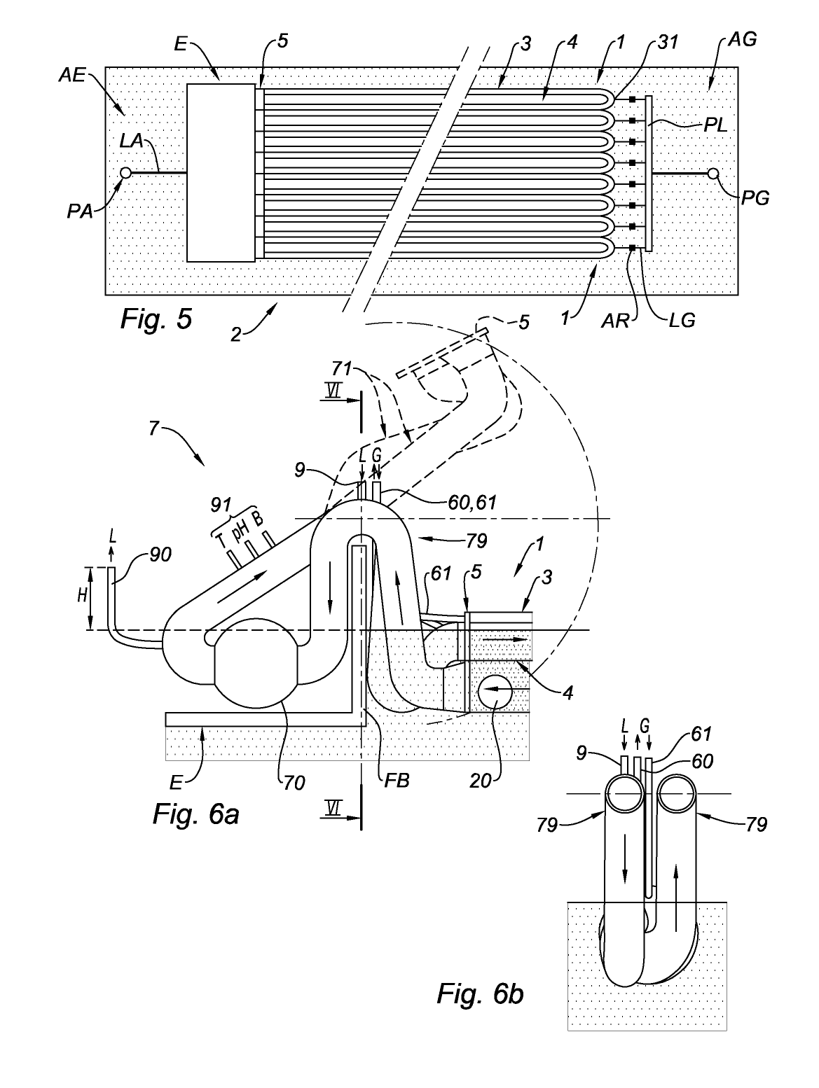

In another embodiment illustrated in FIG. 5 the distal ends 31 of the outer claddings 3 are moored to the bottom of the body of water which here is fairly shallow, via a mooring AG of the outer claddings 3 so that the outer claddings 3 of the casings 1 are no longer able to align themselves with the wind-induced current field

This mooring AG comprises lines LG linking the distal ends 31 of the outer claddings 3 with a common spreader bar PL perpendicular to the outer claddings 3 that are deployed and float horizontally on the water, and a mooring point PG in the form of a beam or pole in particular planted vertically into the bottom of the body of water, with which is the spreader bar PL is connected. These lines L advantageously comprise rotating joints AR, in particular of swivel type, so that the outer claddings 3 are able to rotate freely about their respective longitudinal axes as described above.

The adjustment of the length of the lines LG and their parallel arrangement allows the uniform distribution of mooring tensions between all the outer claddings 3 and allows the rotation or oscillation thereof in order to clean their respective outer walls 31 as described above; the rotation of the outer claddings 3 being made possible by the rotating joints AR on these lines LG between the distal ends 31 of the outer claddings 3 and the spreader bar PL.

If the body of water is open to navigation, daytime and night-time signaling means conforming to local laws and regulations can equip the different points of this floating assembly and in particular the distal ends 31 of the outer claddings 3.

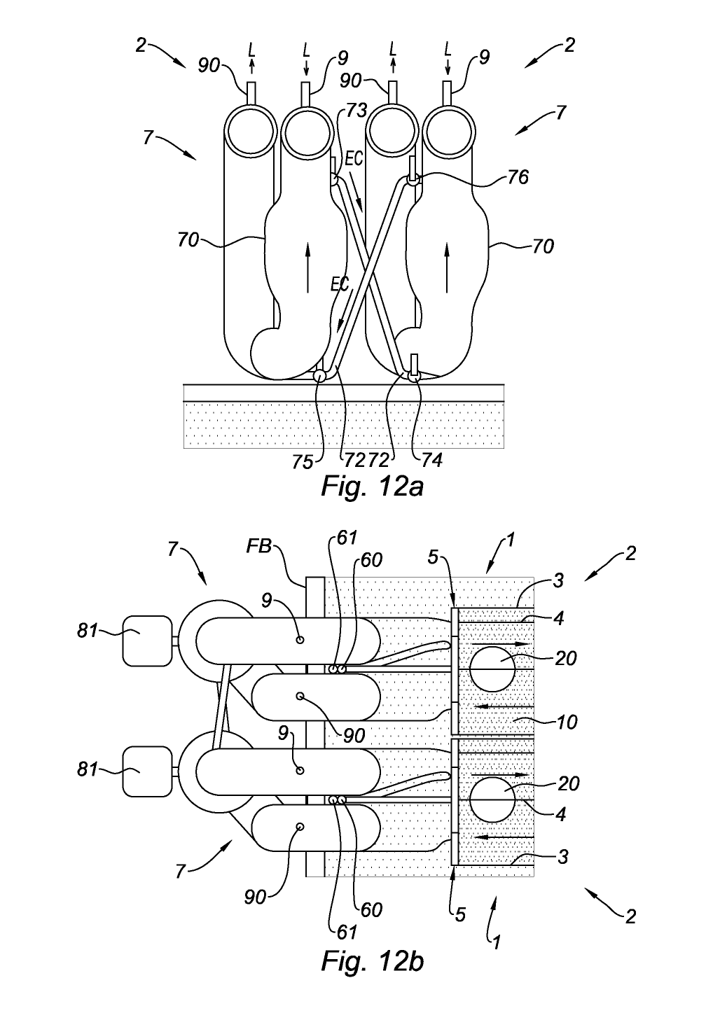

In the embodiments illustrated in FIGS. 6, 7 and 12, the return pipe 7 has a housing 70 (visible in FIGS. 6a, 7, 12, and 13a), of widened cross-section, intended to receive in part the circulation means 8. This housing 70 extends in a main horizontal direction as can be seen in FIG. 6a, or vertical as seen in FIGS. 7 and 12.

In the embodiment illustrated in FIGS. 7a to 7d, the circulation means 8 are in the form of mechanical propelling means which comprise a propeller 80 driven in rotation by a rotary motor 81 via an output shaft 82 of the said motor 81. The motor is arranged outside the reactor 2 and the output shaft 82 passes sealingly through the return pipe 7 to open into the inside of the housing 70 and support the propeller 80 which is thus mobile in rotation inside this housing 70.

Advantageously the housing 70 of the propeller 80 is arranged between a divergence zone and a convergence zone of the return pipe 7 of the liquid culture medium L to ensure hydraulic continuity without any sudden variation in velocity, for the purpose of limiting head losses, accelerations and shear forces suffered by the microorganisms.

According to one advantageous characteristic and as illustrated in FIGS. 7 and 12, the housing 70 is arranged in a vertical upward branch of the return pipe 7 and therefore the propeller 80 has a vertical axis of rotation, to allow evacuation of the gas G which may form inside the housing 70, thereby avoiding cavitation phenomena.

Additionally, as can be seen in FIG. 7, the arrangement of the escape means 60 for the gas G upstream of the circulation means 8, or of the propeller 80, combined with the arrangement of the circulation means 8 in the housing 70 upstream of the gas injection means 61 is also advantageous to avoid circulation of the gas through the propeller 80 which may be detrimental to the functioning thereof. The presence of gas G hampers the functioning of most mechanical propelling means and propellers in particular, and a gas accumulation must therefore be avoided to prevent cavitation of the propeller 80.

In the embodiments illustrated in FIGS. 6, 7 and 11, the return pipe 7 has two curved portions 79 respectively an outward portion and a return portion arranged either side of the housing 70 receiving the circulation means 8. These curved portions 79, or swan-neck portions, have a curvature of substantially 180° to reach over the wall or freeboard FB of the embarkation E; said cleared crossing guarantees the isolating of the circulation means 8 from the water and also the safety of the reactor and of persons using this reactor.

Also, as illustrated in FIGS. 6a and 11, these curved portions 79 can be partly moved to a raised position to move out of the water and lift up the connection plate 5 of the claddings 3, 4, in particular to allow the placing in position of the claddings 3, 4 out of the water under asepsis conditions.

The placing in position of the flexible claddings 3, 4 is described below with reference to FIGS. 11a and 11b. The claddings 3, 4 can be delivered in the form of bobbins B on which a float F is positioned in the center of the bobbin B for stabilization thereof floating on the body of water.

As can be seen in FIG. 11a, the two claddings 3, 4 are mounted on the plate 5 at their respective proximal ends 30, 40; the said plate 5 advantageously occupying a raised position as described above. This operation for securing the claddings 3, 4 to the plate 5 is made under aseptic conditions to avoid introducing contaminants into the culture medium.

As can be seen in FIG. 11, the plate 5 is lowered and partly immersed in the water, the claddings 3, 4 are deployed by filling with liquid L and gas G sterile media. The input of the liquid and gas can be expediently be made using all the openings 11, 12 arranged in the plate 5. If not, only the cladding receiving the input of liquid L and gas G receives the fluids and undergoes the deployment forces. Once the claddings 3, 4 have been unrolled and inflated, the circulation of the liquid medium L can be set up in the reactor 2 by setting in operation the circulation means 8.

A trap TR can be provided on the embarkation E to isolate the outer part of the reactor from the inner part of this reactor 2 once the claddings 3, 4 have been unrolled and inflated.

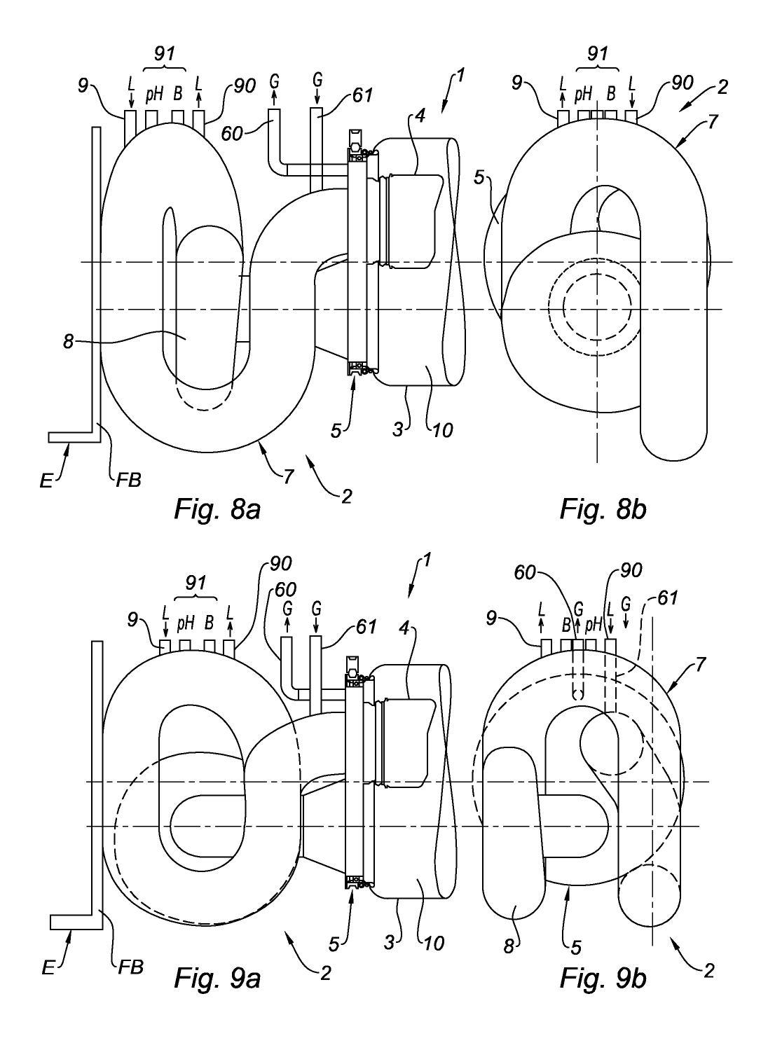

In the embodiments illustrated in FIGS. 8 and 9 the circulation means are in the form of a centrifugal pump 8, the assembly being arranged outside the embarkation E; in this case the term outboard solution is used which has the advantage of being compact but having the drawback of being aggressive for the microorganisms. The passing of cleaning bodies 20 described below requires a return pipe 7 having an open impeller and a volute chamber of large diameter which reduce efficacy. In the embodiment illustrated in FIG. 8 the volute is of frontal volute type and in the embodiment illustrated in FIG. 9 the volute is of sagittal volute type. In these embodiments, with centrifugal pump the plate 5 is secured to an embarkation E or quayside and may optionally be raised to facilitate the placing in position of the claddings 3, 4 out of the water.

As can be seen in FIGS. 1, 6a, 7a, 7d and 12b, the reactor 2 may also comprise one of more cleaning bodies 20 conformed to circulate along the flow pathway, in other words inside the inner cladding 4, the inter-cladding space 10 and the return pipe 7 to clean the inside of the claddings 3, 4 and of the return pipe 7. So that they can circulate in a loop in the reactor 2 the cleaning body or bodies 20 are also conformed to pass through the circulation means 8 for the liquid culture medium, for example through the blades of the propeller 80 in the particular embodiment described above with reference to FIG. 7.

The cleaning body or bodies 20, preferably spherical, have a diameter that is for example substantially equal to the inner diameter of the return pipe 7 to optimize the cleaning of the inner walls of the return pipe 7.

The cleaning body or bodies 20 are also intended to complete the cleaning of the inner wall 34 of the outer cladding 3 and the outer wall 43 of the inner cladding 4 that takes place via the free movement of the inner cladding 4 inside the outer cladding 3; this free movement being promoted by the rotating attachment of the outer cladding 3 on the plate 5.

The difference in velocities between the gas circulation and the liquid circulation has a direct influence on gas/liquid mass transfers and must advantageously be maintained at the highest level possible. This is why each cleaning body 20 must not prevent the passing of the gas. For this purpose, each cleaning body 20 is conformed to allow the passing at least in part of the gas circulating inside the reaction pipe 2 whilst being adapted to be driven by the circulation of the liquid culture medium so that the cleaning body 20 does not have any influence on the difference in velocities between the gas and the liquid medium.

For this purpose, the or each cleaning body 20 is in the form of a brush a spherical brush in particular, comprising an assembly of hairs, bristles or strands or equivalent with a central part carrying these hairs. Therefore in the horizontal claddings 3, 4, the emerging hairs allow the gas to pass in the gas headspace and the central immersed part carrying the hairs is of sufficiently large diameter to form an obstacle to the passing of liquid so that the liquid medium carries the cleaning body 20 with it.

Similarly, the cleaning body 20 can be made in the form of a hollow sphere in elastomer material of which a substantial part of the surface is pierced with holes which allow the gas to pass.

The cleaning body 20 must also be of greater density than water so that it remains in contact with the floor of the claddings 3, 4 as illustrated in FIG. 1; the floor of a cladding corresponding to its lower or bottom part fully immersed.

To avoid any wedging, the spherical cleaning body 20 must have a diameter Dc which does not exceed the maximum value Dcmax corresponding to the difference in diameters of the claddings 3, 4, which translates as the following relation R2: Dcmax=De-Di.

If the above-defined relation R1 is heeded this then gives: Dcmax=(2^{1/2}1). Di.

Owing to the rotation of the outer cladding 3, the inner cladding 4 tends to be driven towards the submerged side of the outer cladding 3. It is therefore preferable that the diameter Dc of the cleaning body 20 should be substantially smaller than the limit value Dcmax defined above so that the cleaning body 20 is able to be contained in the bottom of the inter-cladding space 10, in other words on the floor of the outer cladding 3.

By proceeding in this manner it is ascertained that irrespective of the direction of rotation of the outer cladding 3, the cleaning body 20 is driven towards the larger passages and not the narrower passages, which reduces the risks of the cleaning body 20 being wedged between the inner cladding 4 and the outer cladding 3.

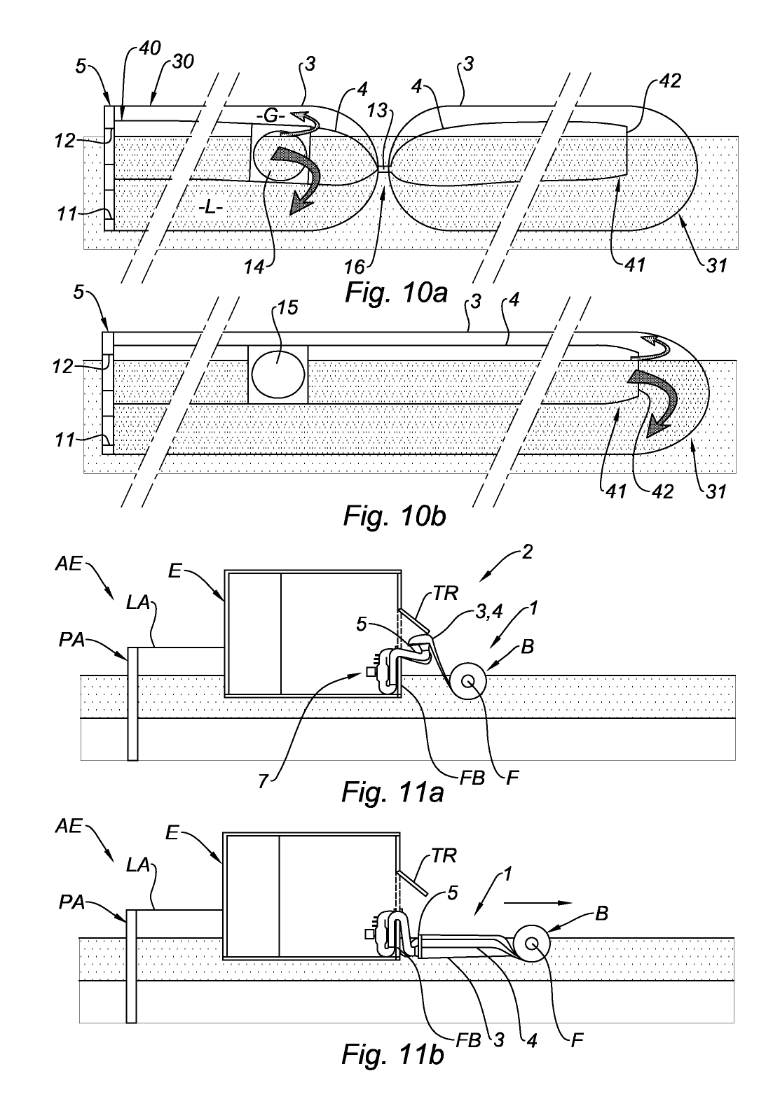

In one particular embodiment of the invention illustrated in FIGS. 10a and 10b, the casing 1 further comprises:

removable clamping or ligature means 13 designed to clamp or ligature the two claddings 3, 4 over an intermediate zone 16 located between the respective proximal ends 30, 40 and distal ends 31, 41 of the claddings 3, 4;

at least one intermediate communication orifice 14 between the inside of the inner cladding 4 and the inter-cladding space 10, the said intermediate orifice 14 being provided on the inner cladding 4 between its proximal end 40 and the intermediate clamping or ligature zone 16; and

means 15 for closing the intermediate orifice 14 in particular of trap type, the said closing means 15 being mobile between an open position (illustrated in FIG. 10a) and a closed position (illustrated in FIG. 10b).

Therefore the inner cladding 4 comprises a trap 15 which can be handled from outside the outer cladding 3 to open or close this intermediate orifice 14.

The intermediate clamping and ligature zone 16 is arranged at about one tenth of the length of the outer 3 or inner 4 cladding, so as only to use a reaction sub-volume corresponding to that portion of the casing 1 and claddings 3, 4 located between the plate 5 and this intermediate zone 16 and which may represent about 1/10 of the total volume of the casing 1; this ratio corresponding to the ratio of inoculation volume to culture volume conventionally used.

At a first stage as illustrated in FIG. 10a, the clamping or ligature means 1 clamp or ligature the two claddings 3, 4 over an intermediate zone 16 located beyond the intermediate orifice 14, thereby isolating the reaction sub-volume. The claddings 3, 4 are filled with sterile nutrient medium and gas and the trap 15 arranged in the inner cladding 4 is open so as to allow the passing of the gas G and liquid L to the end of this reaction sub-volume. In this manner this sub-volume has all the functionalities of the whole volume and is able to function independently. It is therefore possible to inoculate and place in culture.

At a second stage illustrated in FIG. 10b, when the concentration has reached a sufficient level in this sub-volume, the clamping or ligature means 13 are removed and the trap 15 is closed, so that the remainder of the volume is placed in line and on this account is inoculated by the sub-volume; the reactor then enters into full production capacity.

Some cultures must not be exposed to full light for as long as their concentration is insufficient for protection thereof by self-shading. For these types of cultures, it is therefore preferable to ensure protection against light for a period of a few days after inoculation.

To ensure this protection, one solution comprises covering the outer cladding 3 with a longitudinal shadow strip not illustrated) made of opaque material or having strong light absorbance. Therefore the luminosity is greatly attenuated inside the casing 1 when the shadow strip is inserted between the culture and the sunlight.

Two embodiments of the shadow strip can be envisaged with in a first embodiment a first shadow strip and in a second embodiment a second shadow strip.

In the first embodiment, the first shadow strip is added and temporarily positioned on the outer cladding 3 and more specifically on the emerged part (out of the water) of the outer cladding 3. The first shadow strip may fully cover the emerged surface of this outer cladding 3 above the waterline and, for example, take up between one quarter and one third of the total perimeter of the outer cladding 3 (depending on the position of the waterline).

This first shadow strip is positioned fixedly on the outer cladding 3 for the first days of culture when the rotation of the outer cladding 3 is interrupted. Once the concentration of the culture has reached the threshold value defined for the cultivated species, the rotation of the outer cladding 3 is initiated and the first shadow strip can be removed.

In the second embodiment, the second shadow strip is secured to the outer cladding 3 and is therefore directly integrated into the outer cladding 3. The second shadow strip can be fixed onto the outer cladding 3 by gluing or welding, or as a variant the second shadow strip may be in the form of a layer of material (e.g. a paint film or film of opaque material) fixedly deposited on the outer cladding 3. This second shadow strip takes up between one quarter and one third for example of the total perimeter of the outer cladding 3.

This second shadow strip may fully cover the emerged surface of this outer cladding 3, above the waterline, for the first days of culture when the rotation of the outer cladding 3 is interrupted. Once the concentration of the culture has reached the threshold value defined for the species being cultivated the rotation of the outer cladding 3 is initiated and the second shadow strip remains in place on the outer cladding 3.

To prevent this rotation from placing the second shadow strip between the sun and the culture which would translate as lost production, the rotation can start with one half-turn which brings the second shadow strip to the lower immersed part in other words under the waterline, at the floor of the outer cladding 3). Then rotation is continued but not completely (i.e. not a full turn and not even a half-turn) and organized alternately in one direction and then in the other in order to maintain the second shadow strip constantly in the lower immersed part of the outer cladding 3 through which less light passes. In this case, it is necessary to determine a compromise between cleaning of the claddings 3, 4 and exposure to light, since a partial rotation only cleans one part of the circumference of the outer cladding 3, whilst a complete rotation reduces production by repeated attenuation of light owing to the presence of the second shadow strip. Partial rotation over three quarters or two thirds of the circumference could form an advantageous compromise solution.

As illustrated in FIGS. 12a and 12b, the invention also concerns an assembly of photosynthetic reactors comprising at least two reactors 2 conforming to the invention, namely a first (on the left) and a second (on the right) reactor and comprising at least one link line 71, 72 ensuring fluid connection between the first reactor and the second reactor and at least one valve 77, 78 arranged on the said link line 71, 72, to allow the inoculation of one reactor by the other. It is thus possible to interconnect two reactors so that their contents are exchanged, to make possible the inoculation of one reactor by the other whose concentration has reached an advanced stage.

In the embodiment illustrated in FIGS. 12a and 12b, the assembly comprises two link lines 71, 72 between the two reactors 2. The link lines at their respective ends are provided with valves 73, 74 respectively for link line 71, and 75, 76 respectively for the link line 72.

The first link line 71 connects an inlet point arranged on the first reactor 2 downstream of the housing 70 receiving the circulation means, such as the rotating propeller (not shown), to an outlet point arranged on the second reactor upstream of the housing 70 receiving the circulation means of tis second reactor 2.

The second link line 72 connects the inlet point arranged on the second reactor 2 downstream of the housing 70 receiving the circulation means such as a rotating propeller (not shown), to an outlet point arranged on the first reactor upstream of the housing 70 receiving the circulation means of this second reactor 2

The reactors 2 are assembled in parallel to form a coherent production assembly. To make possible the inoculation of one reactor by its neighboring reactor whose microorganism concentration has reached an advanced stage, the assembly provides for interconnecting these two reactors with the link lines 71, 72 so that their respective contents are mixed.

In addition, as can be seen in FIG. 12a, the outlet points of the link lines 71, 72 are placed at the end of the convergence zones located upstream of the corresponding housings 70 to benefit from a Venturi effect.

The valves 73, 74, 75, 76 allow the aseptic connecting of the two link lines 71, 72 which cross connect symmetrically the inlet points and outlet points of the two reactors 2 to be interconnected. The valves 73, 74, 75, 76 are arranged substantially at the inlet and outlet points of the corresponding link lines 71, 72.

The use of said assembly can be made in the following manner to proceed with inoculating the second reactor 2 (on the right) from the first reactor 2 (on the left) already in service, when the concentration of microorganisms has reached the production level.

At a first stage the valves 73, 74 and their counterparts 75, 76 are closed, the first reactor 2 is in service with circulation being set up inside this first reactor, and the second reactor 2 to be inoculated is filled with sterile nutrient medium.

At a second stage, the circulation is set up inside the second reactor 1B and the valves 73, 74 and their counterparts 75, 76 are open to set up a cross exchange between the two reactors as illustrated by the arrows EC in FIG. 12a.

After opening the valves 73, 74 and their counterparts 75, 76, the concentrations become substantially equal in both reactors 2 and it is possible to isolate them by closing the valves 73, 74 and their counterparts 75, 76. To reduce the time of this exchange, a pump (not shown) can be inserted on one and/or the other of the link lines 71, 72.

As illustrated in the FIGS. 13a and 13b, the invention also concerns a reactor 2 comprising a plurality of reaction casings 1 connected in parallel on the connection plates 5, this reactor 2 comprising one and the same return pipe 7 having a housing 70 receiving circulation means 8 such as a propeller 80/rotary motor 81 assembly, and a plurality of conduits in fluid connection with the casings 1.

In this particular form of embodiment, which comprises interconnecting reaction casings 1 in parallel, the advantage is to place in common some functionalities such as the circulation means, the regulation means, but with the drawback of increasing exposure to accidents, in particular contaminations.

In this embodiment, the return pipe 7 comprises a collecting conduit 66 in which the housing 70 is provided which receives the circulation means 8 and a plurality of distribution conduits 67 connected firstly to the collecting conduit 66 and secondly to the respective casings 1 so that the liquid medium is collected on leaving the casings 1, passes in the circulation means and is then distributed at the inlet to the casings 1. Expediently, the distribution is made in the same order as collection so that the flow rates in the casings 1 are uniform.

Expediently, and to maintain the velocities as uniform as possible the collecting conduit 66 and the distribution conduits 67 are of variable section decreasing from one end to the other of the channel that they form.

The culture method of photosynthetic microorganisms, algae in particular using a reactor 2 conforming to the invention comprises the following steps:

injecting a liquid culture medium into the reaction casing 1 at a flow rate controlled by the liquid injection means 9;

injecting a gas G into the reaction casing 1 at a flow rate controlled by the gas injection means 61;

pressurizing the outer cladding 3 of the casing 1 comprising setting up an inflation overpressure in this outer cladding 3 to ensure the buoyancy of this outer cladding 3 and the deployment thereof;

circulating the liquid culture medium with the circulation means 8;

controlling these circulation means 8 and the gas injection means 61 to set up a biphasic flow pattern in the reaction casing 1 of the gas/ liquid culture medium, of stratified flow type or slug flow or elongated bubble flow type; and

recovering the photosynthetic microorganisms with the outlet pipe 90.

Throughout the pathway in the casing 1, the liquid medium containing the photosynthetic microorganisms receives sunlight through the transparent material of the claddings 3, 4, exchanges heat with the body of water by diffusion, by mixing and by conduction through this same material, and exchanges components with the gas G via their common interface. The production capacity depends especially on the length of the claddings 3, 4 of the casing 1; several casings 1 able to be usefully arranged in number alongside one another to ensure the essential cleaning function described above.

Advantageously, the rate of circulation of the gas is set at about 0.5 and 1.5 m/s, corresponding to an adequate velocity for flow rates needed for the reaction.

Further advantageously, the means for setting in circulation 8 comprise a propeller 80 driven in rotation by a motor 81 and the speed of rotation of the propeller 80 is less than 100 rotations per minute to limit mechanical stresses within the liquid culture medium.

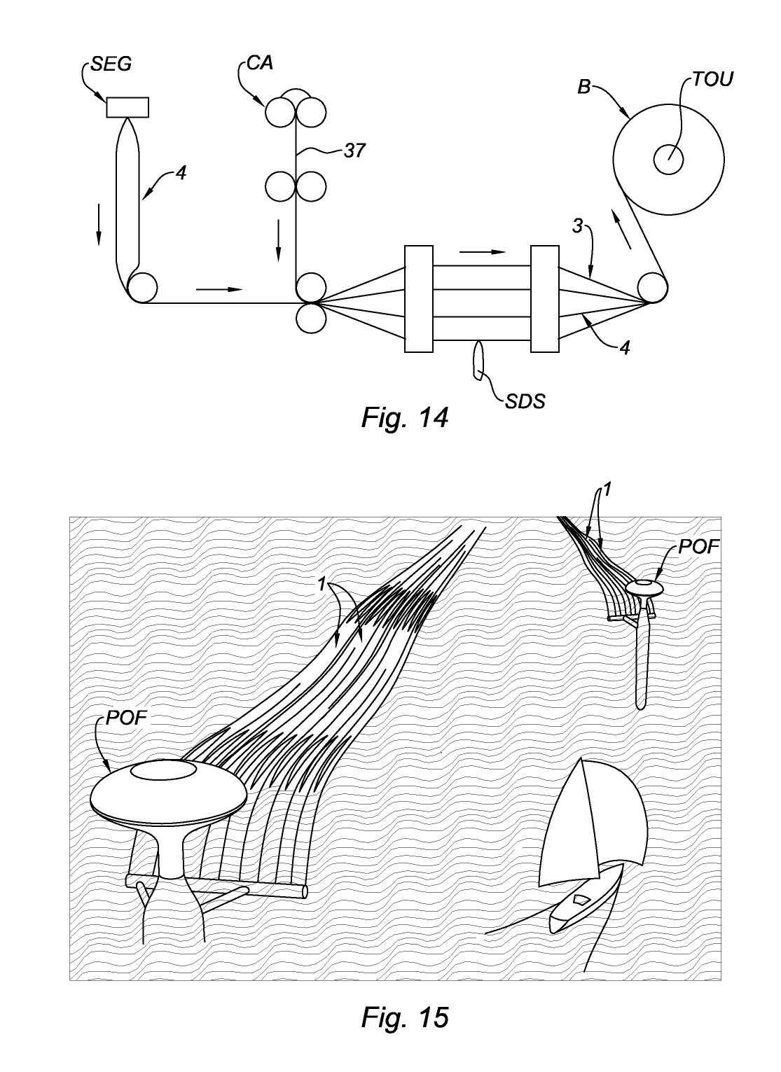

The invention also concerns a method for manufacturing a casing 1 described with reference to FIG. 14 comprising the following steps:

fabricating the inner cladding 4 using an extrusion method of plastic material and blowing the extruded plastic, otherwise called extrusion-blowing process using a SEG blown film extrusion system;

fabricating an outer sheet 37 in plastic material, in particular using a calendaring process and calendar CA;

wrapping the outer sheet 37 round the inner cladding 4 until the two opposite longitudinal edges of the outer sheet 37 meet;

welding the outer sheet 37 along its two opposite edges joined at the wrapping stage using a SDS welding system so as to form the outer cladding 3 surrounding the inner cladding 4 ; and

winding the two claddings 3, 4 one in the other on a single reel TOU so as to form a bobbin B.

The claddings 3, 4 are produced and arranged one in the other in the workshop before delivery. This method meets the problem of inserting one cladding of long length into another by proposing simultaneous, continuous fabrication of these two claddings 3, 4, preferably aseptically so as to reduce risks of initial contamination of the cultures.

This manufacturing process which comprises closing the outer cladding 3 around the inner cladding 4 allows this inner cladding 4 to be equipped during assembly in particular with the communication orifice 42 and optionally the intermediate orifice 14 provided with its trap 15.

The winding of the two claddings 3, 4 arranged one in the other starts at their distal ends before the deployment or unrolling described above with reference to FIGS. 11a and 11b. The outer cladding 4 is closed at its distal end as described above, and this distal end is optionally equipped with a line hooking system such as the line LG described above with reference to FIG. 5.

Expediently, the thickness of the constituent film of the claddings 3, 4 may increase from the distal end to the proximal end to increase resistance at the same time as forces are applied to the film.

Evidently, the example of embodiment described above is in no way limiting and other improvements and details can be made to the casing, to the reactor and to the methods of the invention without departing from the scope of the invention, wherein other forms of outer cladding and/or inner cladding and/or connection plate and/or return pipe can be provided for example.

It can therefore be envisaged as schematically illustrated in FIG. 15 to make provision for mooring of the casings 1 onto an offshore platform POF on high seas. This offshore platform POF forms a service floating structure which may for example house an operating team and hence the functioning is preferably fully automated.

The offshore platform POF is preferably formed by a spar buoy system very little sensitive to surface agitation. In this case, the immersed part of the buoy contains the buoyancy reserves in the upper part, the ballast in the lower part and the functionalities and storage at the intermediate levels.

Provision may also be made on this offshore platform POF for a structure carrying the connecting plates for the claddings of the casings 1 which is linked to the offshore platform POF by one or more arms whose angle can be adjusted; this structure carrying the connector plates can therefore be brought to the surface for maintenance on the plates, or immersed in normal operating mode.

Other activities such as fish breeding can be associated with this offshore platform POF. Confinement traps for fish breeding can be placed for example underneath the expanse of casings 1 floating on the surface of the sea.

In addition, it can also be envisaged that the offshore platform POF may be provided with propelling means, optionally automated, so that it can move to follow the surface temperatures that are best adapted to the culture of the photosynthetic microorganisms, escaping from the most unfavorable weather conditions and avoiding collision. The movements of the offshore platform PF are controlled in situ or from the land in relation to weather and radar information to search for the best routes. The offshore platform POF may also draw close to the shore to unload production and to receive supplies.

1.-18. (canceled)

19. A reaction casing for a photosynthetic reactor for a culture of photosynthetic microorganisms, algae in particular, the reaction casing being designed firstly to float on a body of water and secondly to delimit a biphasic flow pathway for a gas/liquid culture medium between a first and a second opening of the reaction casing, the reaction casing comprising two claddings, respectively outer and inner, made at least in part of a material transparent to light rays, the inner cladding extending inside the outer cladding so that the claddings delimit between them an inter-cladding space in fluid connection with the first opening of the reaction casing, wherein the outer cladding has an open proximal end and a closed distal end and in that the inner cladding has an open proximal end in fluid connection with the second opening of the reaction casing and a distal end provided with at least one communication orifice between the inside of the inner cladding and the inter-cladding space.

20. The casing according to claim 19 wherein at least one of the two claddings, respectively outer and inner, is made in a flexible material adapted to allow folding, inflation, transverse deformation and/or bending of the cladding.

21. The casing according to claims 19 further comprising:
an external connection part on which the proximal end of the outer cladding is hermetically mounted, and on which the first opening of the reaction casing is arranged in fluid connection with the inter-cladding space; and
an internal connection part on which the proximal end of the inner cladding is hermetically mounted and on which the second opening of the reaction casing is arranged in fluid connection with the proximal end of the inner cladding.

22. The casing according to claim 21 wherein the proximal end of the inner cladding is mounted in rotation on the internal connection part.

23. The casing according to claim 21 wherein the internal connection part is mounted inside the external connection part.

24. The casing according to claim 21 wherein the external connection part comprises coupling means with means for driving in rotation the external connection part to drive the outer cladding in rotation.

25. The casing according to claim 23 wherein the internal connection part is free in rotation in the external connection part, so that the rotation of the outer cladding ensures the rotation of the inner cladding by friction between the two claddings.

26. The casing according to claim 19 further comprising a third cladding in flexible material extending inside the inner cladding to allow the injection or extraction of gas at the distal ends of the two claddings, respectively outer and inner.

27. The casing according to claim 20, further comprising:
removable clamping or ligature means designed to clamp or ligature the two claddings over an intermediate zone located between the respective proximal and distal ends of the two claddings;
at least one intermediate communication orifice between the inside of the inner cladding and the inter-cladding space, the intermediate communication orifice being provided on the inner cladding between its proximal end and said intermediate clamping or ligature zone; and
means for closing the intermediate communication orifice, in particular of trap type, the closing means being mobile between an open position and a closed position.

28. The casing according to claim 19 further comprising a longitudinal shadow strip partly covering the outer cladding and made iin materal that is opaque or having strong light absorbance, the shadow strip being added and positioned temporarily on the outer cladding or secured onto the outer cladding.

29. A photosynthetic reactor adapted for a culture of photosynthetic microorganisms, algae in particular, comprising:
at least one reaction casing according to claim 19;
at least one return pipe ensuring fluid connection between the first and second openings of the reaction casing;
at least one circulation means arranged in the return pipe and designed to set the liquid culture medium in circulation in the return pipe and in the reaction casing;
at least one liquid injection means arranged in the return pipe and designed to allow the injection of liquid into the reaction casing;
at least one gas injection means arranged in the return pipe and designed to allow the injection of gas into the reaction casing; and
at least one gas escape means arranged in the return pipe and designed to allow the escape of gas injected into the reaction casing.

30. A method for the culture of photosynthetic microorganisms, algae in particular, using a reactor according to claim 29 and comprising the following steps:
injecting a liquid culture medium into the reaction casing at a controlled flow rate using the liquid injection means;
injecting a gas into the reaction casing at a controlled flow rate using the gas injection means;
setting the liquid culture medium in circulation with the circulation means;
controlling the circulation means and gas injection means to set up in the reaction casing a biphasic flow pattern of the gas/liquid culture medium of stratified flow, or slug or elongated bubble flow type.

31. The method according to claim 30 wherein the control step comprises a step to control the rate of circulation of the liquid in the reaction casing at between about 0.1 and 1.0 m/s.

32. The method according to claims 30 wherein the circulation means comprise a propeller driven in rotation by a motor, and wherein the speed of rotation of the propeller is less than about 1000 rpm.

33. A method for manufacturing a reaction casing according to claim 19 comprising the following steps:
fabricating the inner cladding, in particular by extruding a plastic material and blowing the extruded plastic;
fabricating an outer sheet in plastic material in particular by calendering;
wrapping the inner cladding with the outer sheet as far as the junction of two opposite edges; and
welding the outer sheet along its two opposite edges joined at the wrapping stage, so as to form the outer cladding surrounding the inner cladding.
